(19) United States
(12) Patent Application Publication
(10) Pub. No.: US 2016/0120420 A1
(43) Pub. Date: May 5, 2016

(54) VASCULAR MEASUREMENT SYSTEM

(71) Applicants: David A. Liedl, Eyota, MN (US); William D. Davis, Denver, CO (US)

(72) Inventors: David A. Liedl, Eyota, MN (US); William D. Davis, Denver, CO (US)

(21) Appl. No.: 14/528,594

(22) Filed: Oct. 30, 2014

(51) Int. Cl. A61B 5/022; A61B 5/00; A61B 5/026

(52) U.S. Cl. CPC A61B 5/02241; A61B 5/026; A61B 5/7278; A61B 5/6828; A61B 5/6826; A61B 5/6824; A61B 5/6829; A61B 2560/0475

(57) ABSTRACT

A vascular measurement system is provided to perform various types of peripheral vascular measurements to evaluate at least one of arterial blood flow and venous blood flow. The system includes a plurality of ports configured to connect a plurality of inflatable cuffs, an inflation device, a deflation device, and a volume measuring device configured to withdraw a predetermined volume of air contained in at least one of the first and second inflatable cuffs and resupply the predetermined volume of air into the at least one of the first and second inflatable cuffs.

### BACKGROUND

Peripheral vascular disease (PVD) refers to diseases of the blood vessels (arteries and veins) located outside the heart and brain. Although there are many causes of peripheral vascular disease, the peripheral vascular disease is commonly used to refer to peripheral arterial disease (PAD), which develops when the arteries become blocked or narrowed.

Several tests can be used to diagnose peripheral vascular disease. The tests include various non-invasive vascular tests, which utilize various types of technology to evaluate the health of blood vessels at rest and/or with exercise. To perform different peripheral vascular test, different test systems are typically used, which incorporate different technologies. Therefore, medical practitioners or other operators need to be capable of using different test systems to perform different types of peripheral vascular tests.

Further, some of the PVD test systems, such as systems for evaluating venous blood flow, use flowmeters configured to measure mass or volumetric flow rate of a liquid or gas used in the tests. The flowmeters are typically expensive, thereby increasing the cost of the test systems.

### SUMMARY

In general terms, this disclosure is directed to a vascular measurement system. In one possible configuration and by non-limiting example, the system is configured to be multifunctional and performs various types of peripheral vascular measurements. Further, the system includes a reliable, cost-efficient volume measuring device that replaces a flowmeter. Various aspects are described in this disclosure, which include, but are not limited to, the following aspects.

One aspect is a system for determining blood pressure of a subject. The system comprising: a first port configured to be connected to a first inflatable cuff, the first inflatable cuff coupled to a first pressure sensor; a second port configured to be connected to a second inflatable cuff, the second inflatable cuff coupled to a second pressure sensor; an inflation device configured to inflate at least one of the first and second inflatable cuffs; a deflation device configured to deflate at least one of the first and second inflatable cuffs; and a volume measuring device configured to withdraw a predetermined volume of air contained in at least one of the first and second inflatable cuffs and resupply the predetermined volume of air into the at least one of the first and second inflatable cuffs.

Another aspect is an apparatus for evaluating vascular flow. The apparatus comprising: a housing; a plurality of ports arranged on the housing and configured to be connected to a plurality of inflatable cuffs, the plurality of inflatable cuffs coupled to a plurality of pressure sensors; an inflation device configured to inflate at least one of the plurality of inflatable cuffs; a deflation device configured to deflate at least one of the plurality of inflatable cuffs; one or more processing devices within the housing; and a computer readable storage device storing software instructions that, when executed by the one or more processing devices, cause the one or more processing devices to measure either arterial blood pressure of a test subject or venous blood pressure of the test subject from at least a part of the plurality of inflatable cuffs.

Yet another aspect is a method of determining blood pressure of a subject. The method comprising: arranging a test subject in a first position; securing one of a plurality of inflatable cuffs to a limb of the test subject, the plurality of inflatable cuffs coupled to a plurality of pressure sensors; inflating the one of the plurality of inflatable cuffs to a predetermined pressure; recording a pressure from one of the plurality of pressure sensors coupled to the one of the plurality of inflatable cuffs; withdrawing a predetermined volume of air contained in the one of the plurality of inflatable cuffs into a chamber of a volume measuring device by moving a plunger within a barrel of the volume measuring device in a first longitudinal direction; recording a pressure change from the one of the plurality of pressure sensors coupled to the one of the plurality of inflatable cuffs; and calculating a pressure-to-volume relationship from the predetermined volume and the pressure change.

### BRIEF DESCRIPTION OF THE DRAWINGS

FIG. 1 is a diagram of an example system for performing various vascular non-invasive measurements.

FIG. 2 is a state diagram illustrating the operation of a vascular measurement apparatus of FIG. 1.

FIG. 3 is a block diagram illustrating example functional features of the vascular measurement apparatus of FIG. 1.

FIG. 4 is a schematic overview of the vascular measurement apparatus of FIG. 1.

FIG. 5 is a circuit diagram of an example pump driver circuitry for operating an air pump of FIG. 4.

FIG. 6 is a circuit diagram of an example band pass filter used for filters of FIG. 4.

FIG. 7 is a circuit diagram of an example controller and a communications device of FIG. 4.

FIG. 8 illustrates an exemplary architecture of an analyzing computing device of FIG. 4.

FIG. 9 is a diagrammatic view of an example volume measurement device of FIG. 4.

FIG. 10 is a diagram of an example system for performing various vascular measurements.

FIG. 11 illustrates examples of an arterial test mode of FIG. 2.

FIG. 12 is a schematic view of an example placement of inflatable cuffs to perform an ABI test or a PVR test.

FIG. 13 is a schematic view of another example placement of the inflatable cuffs to perform the ABI test.

FIG. 14 is a schematic view of an example placement of the inflatable cuffs to perform an arterial inflow measurement.

FIG. 15 is a flowchart illustrating an example method of performing an ABI measurement with the system of FIG. 1 or 10.

FIG. 16 shows a flowchart to a general sequence of steps performed with each test of the ABI measurement with the system of FIG. 1 or 10.

FIG. 17 is a flowchart illustrating inflation of a sensing cuff in more detail.

FIG. 18 shows a detailed flow chart to inflation of an occluding cuff.

FIG. 19 shows a detailed flowchart to a deflation sequence of the occluding cuff during a sensing phase of a test cycle and deflation of both cuffs upon completion of the test.

FIGS. 20A and 20B show exemplary pressure versus time waveforms for pressures sensed during a test of a limb at the occluding and sensing cuffs.

FIGS. 21A-21C show composite plethsymographic waveforms for a limb under test in FIGS. 20A and 20B.

FIG. 22 illustrates a flowchart of an example method of performing a PVR measurement with the system of FIG. 1 or 10.

FIG. 23 is a flowchart of an example method of performing an arterial inflow measurement with the system of FIG. 1 or 10.

FIG. 24 illustrates example tests in a venous test mode of FIG. 2.

FIG. 25 is a schematic view of an example placement of inflatable cuffs to perform an obstruction test

FIG. 26 is a schematic view of an example placement of the inflatable cuffs to perform an incompetence test and an exercise test.

FIG. 27 shows a system operational timeline relative to a position of a subject and related venous blood flow volume measurements monitored by the system of FIG. 1 or 10.

FIG. 28 is a flowchart of an example method of performing a setup process of FIG. 24.

FIG. 29 is a flowchart of an example method of performing an obstruction test of FIG. 24.

FIGS. 30A-30C illustrate example test results of the obstruction test performed by the method in FIG. 29.

FIG. 31 is a flowchart of an example method of performing an incompetence test of FIG. 24.

FIGS. 32A and 32B illustrate example test results of the incompetence test performed by the method in FIG. 31.

FIG. 33 is a flowchart of an example method of performing an exercise test of FIG. 24.

FIGS. 34A and 34B illustrate example test results of the exercise test performed by the method in FIG. 33.

FIG. 35 illustrates an example result of an ejection fraction test of FIG. 24.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

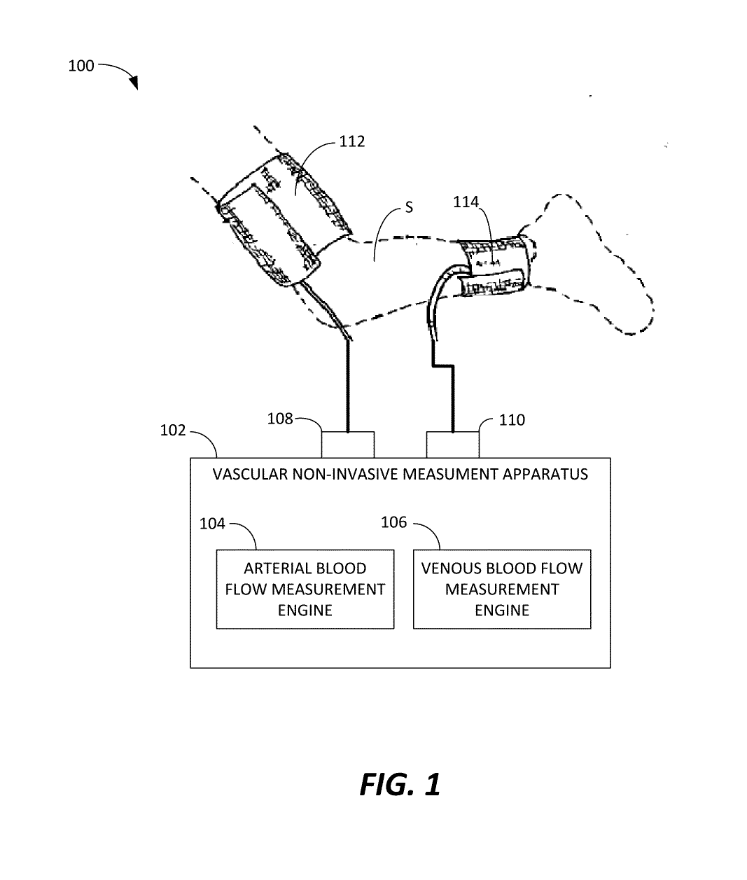

FIG. 1 is a diagram of an example system 100 for performing a variety of vascular non-invasive measurements. In some embodiments, the system 100 includes a vascular measurement apparatus 102 including an arterial blood flow measurement engine 104 and a venous blood flow measurement engine 106. The apparatus 102 further includes a first port 108 and a second port 110. Also shown are a first inflatable cuff 112 and a second inflatable cuff 114, which are secured around a limb of a test subject S.

The vascular measurement apparatus 102 operates to evaluate peripheral vascular blood flow to detect several vascular diseases. Vascular diseases include any conditions that affect the circulatory system of a patient, such as peripheral vascular disease (PVD). Peripheral vascular disease is a progressive circulation disorder and involves disease in any of the blood vessels outside of the heart and diseases of the lymph vessels, such as arteries, veins, or lymphatic vessels.

In some cases, peripheral vascular disease can be diagnosed by evaluating arterial blood flow and/or venous blood flow. Due to differences between arteries and veins in several aspects (e.g., anatomic characteristics and structures), the arterial blood flow and the venous blood flow have been typically measured by different devices that apply different steps of test and technologies.

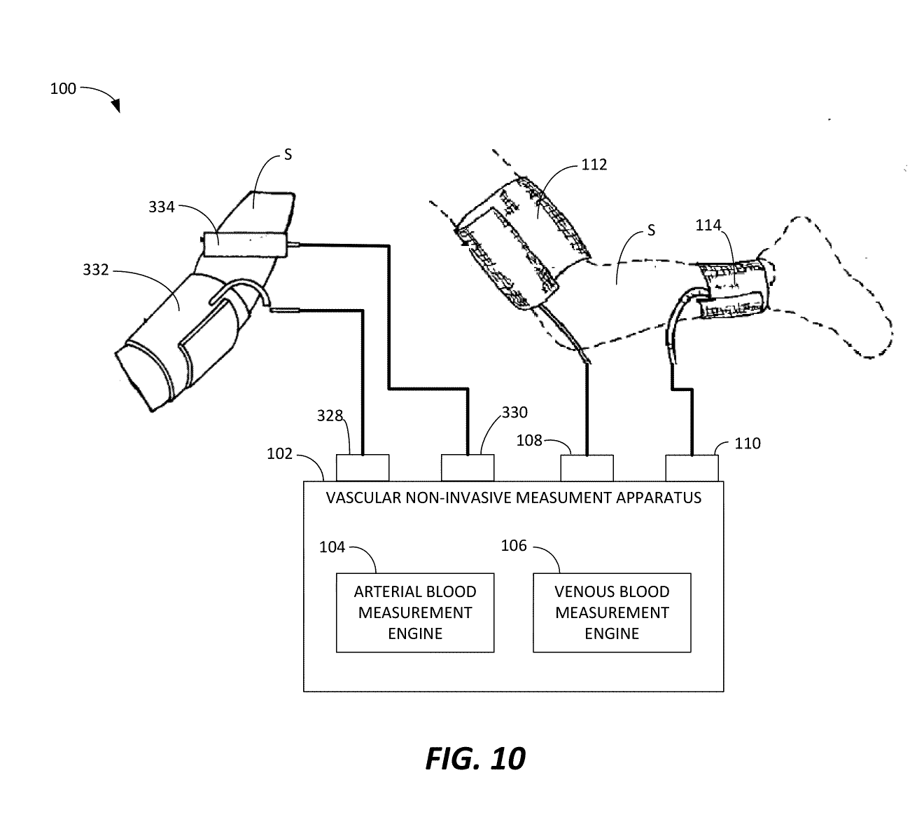

As described, the apparatus 102 is configured to selectively execute either the arterial blood flow measurement engine 104 or the venous blood flow measurement engine 106. In addition, as illustrated in FIG. 10, the apparatus 102 is further configured to simultaneously execute both of the arterial blood flow measurement engine 104 and the venous blood flow measurement engine 106. Accordingly, the apparatus 102 can be used to measure at least one of the arterial blood flow and the venous blood flow, as desired.

The arterial blood flow measurement engine 104 operates to perform various operations for evaluating arterial blood flow in one or more limbs of the test subject S. The arterial blood flow measurement engine 104 is executed by the apparatus 102.

The venous blood flow measurement engine 106 operates to perform various operations for evaluating venous blood flow in one or more limbs of the test subject S. Similarly to the arterial blood flow measurement engine 104, the venous blood flow measurement engine 106 is executed by the apparatus 102.

The first and second ports 108 and 110 are configured to connect the first and second inflatable cuffs 112 and 114 to the apparatus 102, respectively. As described herein, the first and second ports 108 and 110 are not functionally distinguishable and configured to provide the same function and performance. The first and second ports 108 and 110 can therefore be interchangeably used as necessary. Accordingly, the first and second ports 108 and 110 allow selectively using the apparatus 102 for either arterial blood flow test or venous blood flow test, or both. Examples of the ports 108 and 110 are described herein in more detail.

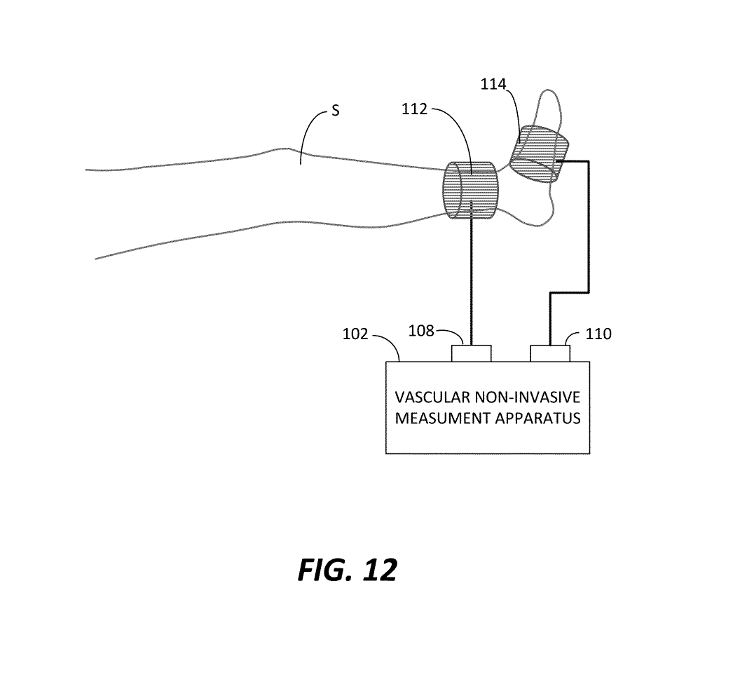

The first and second inflatable cuffs 112 and 114 are configured to be wound and inflated around a portion of a limb of the subject S to press the portion of the limb, thereby restricting blood flow. In the depicted example, the first and second inflatable cuffs 112 and 114 are mounted to the test subject's thigh and calf/ankle However, the first and second inflatable cuffs 112 and 114 can be selectively secured to different locations of one or more limbs of the subject S, depending on a test performed by the apparatus 102. Examples of the inflatable cuffs 112 and 114 are described herein in more detail, and different locations of the cuffs 112 and 114 are illustrated in FIGS. 12-14 and 25-26.

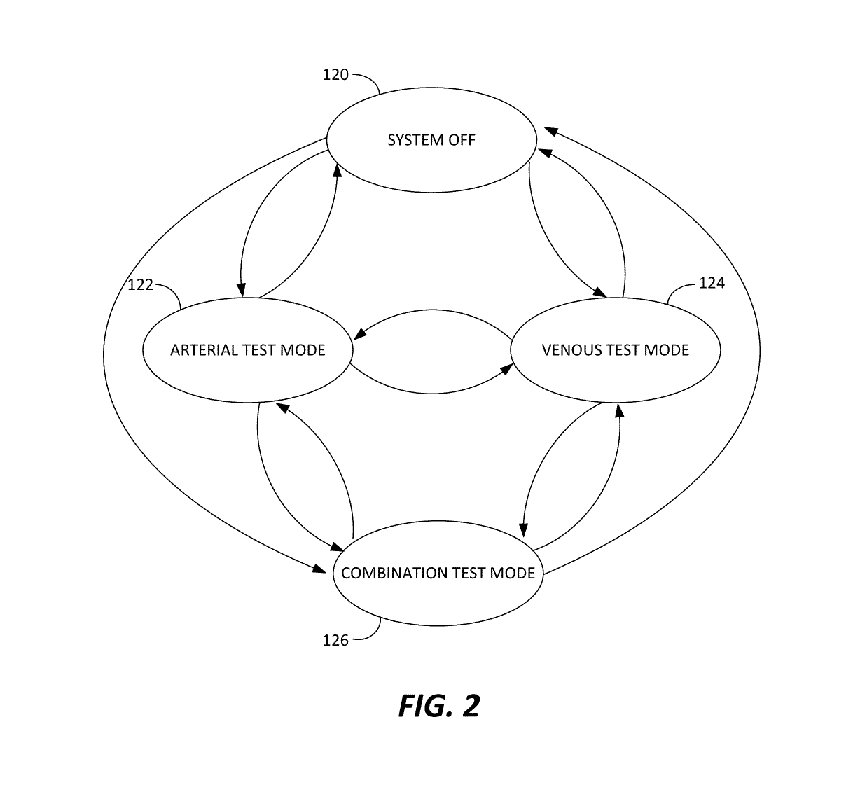

FIG. 2 is a state diagram illustrating the operation of the vascular measurement apparatus 102. As depicted, the apparatus 102 can operate in four different modes: a system off mode 120, an arterial test mode 122, a venous test mode 124, and a combination test mode 126. The four different modes 120, 122, 124 and 126 are interchangeable.

At the system off mode 120, the apparatus 102 is turned off and do not operate. At the arterial test mode 122, the apparatus 102 executes the arterial blood flow measurement engine 104 to perform an arterial blood flow test. At the venous test mode 124, the apparatus 102 executes the venous blood flow measurement engine 106 to perform a venous blood flow test. At the combination test mode 126, the apparatus 102 executes both of the arterial and venous blood flow measurement engines 104 and 106 to simultaneously perform both arterial and venous blood flow tests.

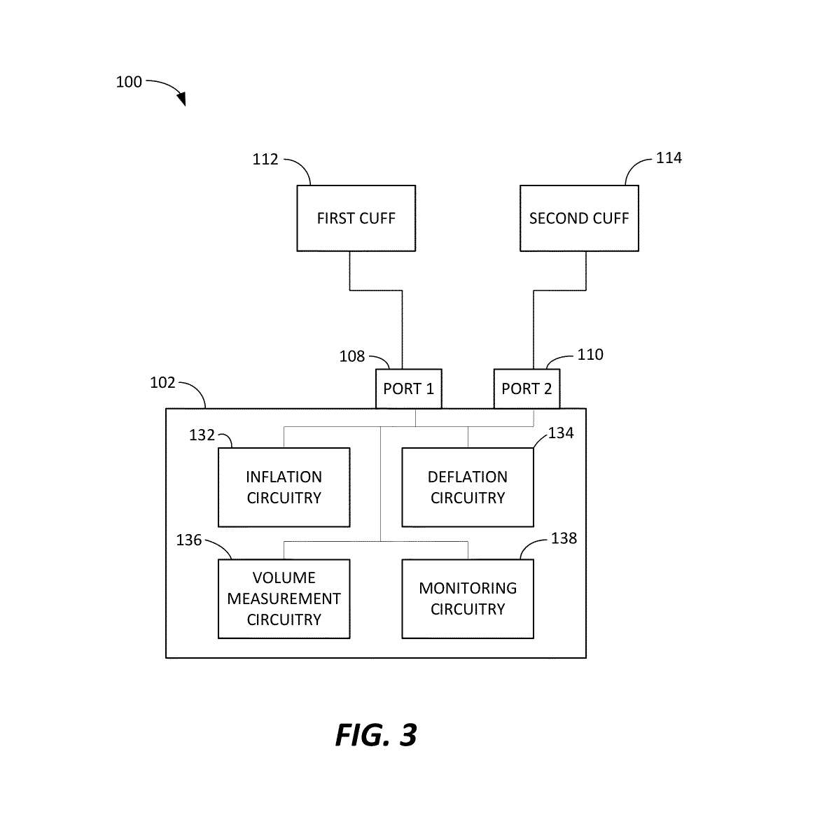

FIG. 3 is a block diagram illustrating example functional features of the vascular measurement apparatus 102 of FIG. 1. In some embodiments, the apparatus 102 includes an inflation circuitry 132, a deflation circuitry 134, a volume measurement circuitry 136, and a monitoring circuitry 138.

The inflation circuitry 132 operates to inflate at least one of the first and second cuffs 112 and 114. The inflation circuitry 132 controls components of the apparatus 102 configured to provide air into at least one of the first and second cuffs 112 and 114.

The deflation circuitry 134 operates to deflate at least one of the first and second cuffs 112 and 114. The deflation circuitry 134 controls components of the apparatus 102 configured to discharge air from at least one of the first and second cuffs 112 and 114.

The volume measurement circuitry 136 operates to establish a pressure-to-volume relationship that is used in evaluating the vascular blood flow as described herein. The volume measurement circuitry 136 is configured to withdraw a predetermined volume of air contained in at least one of the first and second inflatable cuffs 112 and 114 and resupply the predetermined volume of air into the at least one of the first and second inflatable cuffs 112 and 114. In some embodiments, the volume measurement circuitry 136 operates to pull out the predetermined volume of air from one of the first and second inflatable cuffs 112 and 114 and temporarily contain the withdrawn air before refilling the one of the first and second inflatable cuffs 112 and 114 with the withdrawn air.

The volume measurement circuitry 136 is also used to measure the volume of air withdrawn from at least one of the first and second cuffs 112 and 114. As described, the volume of withdrawn air can be calculated without complex mechanism, and the calculated air volume can be used to establish the pressure-to-volume relationship. An example of the volume measurement circuitry 136 is illustrated and described herein in more detail.

The monitoring circuitry 138 operates to monitor and measure pressures detected at the first and second inflatable cuffs 112 and 114. The pressures detected at the cuffs 112 and 114 can represent blood pressures underneath the cuffs 112 and 114. In some embodiments, the pressures detected at the cuffs 112 and 114 can represent the pressure of air contained in the cuffs 112 and 114. For example, when the cuffs 112 and 114 are deflated, a pressure decreases between the cuffs 112 and 114 and the skin of the subject's limb around which the cuffs 112 and 114 are secured. Then, the monitoring circuitry 138 can monitor a pressure difference by detecting a change in pressure between the cuffs and the subject's limb.

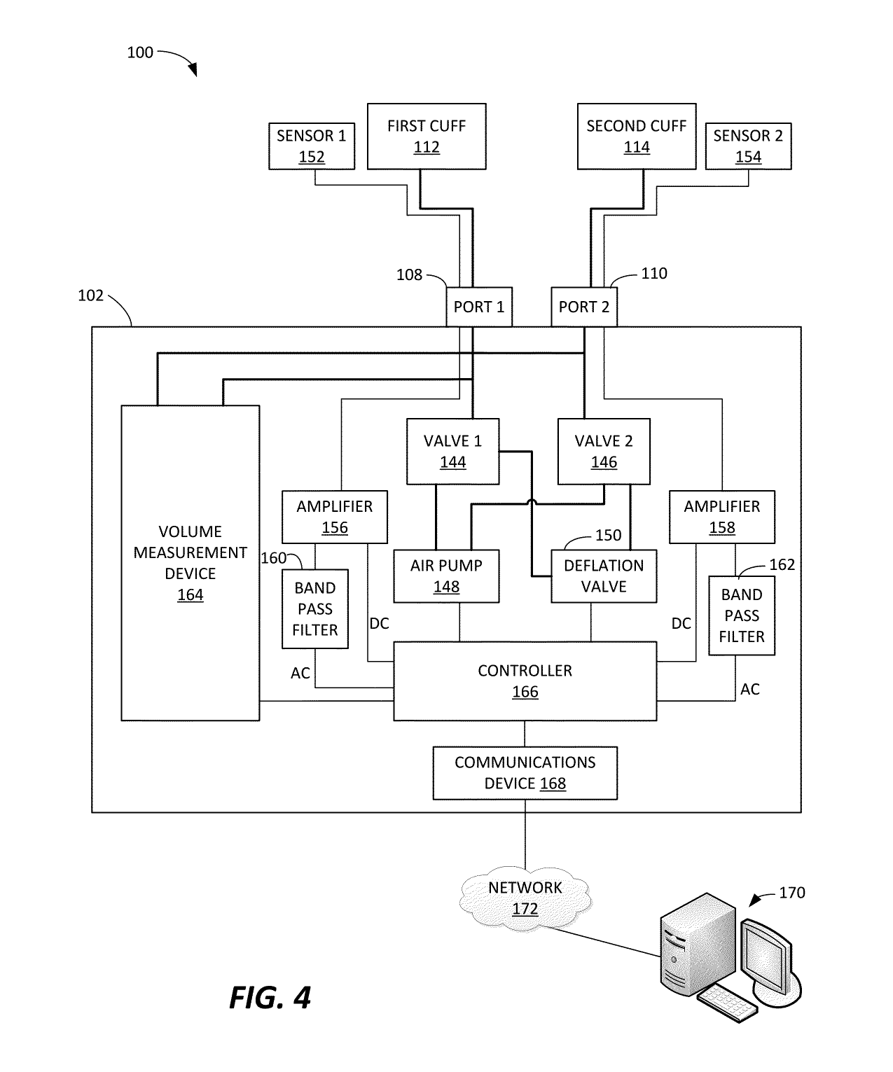

FIG. 4 is a schematic overview of the vascular measurement apparatus 102 of FIG. 1. In addition to the first and second ports 108 and 110 and the first and second inflatable cuffs 112 and 114, in some embodiments, the apparatus 102 further includes one or more conduits 142, first and second valves 144 and 146, an air pump 148, a deflation valve 150, first and second sensors 152 and 154, amplifiers 156 and 158, filters 160 and 162, a volume measurement device 164, a controller 166, and a communications device 168. In some embodiments, the apparatus 102 is configured to communicate with an analyzing computing device 170 through a communication network 172.

The first and second ports 108 and 110 are configured to be connected to the first and second inflatable cuff 112 and 114, respectively, via the conduits 142. As discussed herein, the first and second ports 108 and 110 are configured to have the same functionalities, and thus different types of inflatable cuffs can be interchangeably connected to the ports 108 and 110.

The first and second inflatable cuffs 112 and 114 are configured to be used as a pair of portable, inflatable sensing and occluding cuffs which are respectively constructed to perform sensing and occlusion functions. Each of the first and second inflatable cuffs 112 and 114 can be used as either the sensing cuff or the occluding cuff. As described herein, in the arterial test mode 122 or the venous test mode 124, one of the cuffs 112 and 114 is fitted to the calf or ankle of a test subject S to be used as a sensing cuff, and the other is fitted to the subject's thigh to be used as an occluding cuff. In the arterial test mode 122, one of the cuffs 112 and 114 is fitted to the wrist or finger to be used as a sensing cuff, and the other is fitted to the upper arm to be used as an occluding cuff.

The cuffs 112 and 114 can be constructed to any desired sharp and size to accommodate the limb and task to be performed. In some embodiments, the cuffs 112 and 114 are cloth covered. When used as an occluding cuff, the cuffs 112 and 114 can inflate and deflate over a nominal pressure range sufficient to occlude blood flow in a first portion of the limb (e.g., the subject's leg in the arterial and venous test modes 122 and 124, and the subject's leg or upper arm in the arterial test mode 122). When used as a sensing cuff, the cuffs 112 and 114 can operate at pressures sufficient to retain the cuffs to a second portion of the limb (e.g., the subject's calf/ankle in the arterial and venous test modes 122 and 124, and the subject's wrist/finger in the arterial test mode 122) and maintain sensor contact with the limb.

The cuffs 112 and 114 include appropriate fasteners, such as overlapping hook and loop fasteners, to securely attach to a limb (e.g., upper arm, leg or ankle) or appendage (e.g., wrist, finger or toe). In some embodiments, one of the cuffs 112 and 114 is configured to be slightly smaller than the other cuff to facilitate attachment to the distal sensing regions of the limb extremities (e.g., calves, ankles, toe, wrist, or finger) when used as a sensing cuff. Examples of the cuffs 112 and 114 include a cuff manufactured by the Hokanson Co. (for a occluding cuff) and a CRITIKON™ cuff manufactured by General Electric Co. (for a sensing cuff).

The conduits 142 are configured to connect the first and second cuffs 112 and 114, the first and second inflation valves 144 and 146, the air pump 148, the deflation valve 150, and the volume measurement device 164. As described herein, the conduits 142 selectively provide one or more channels for air flow among the cuffs 112 and 114, the valves 144 and 146, the air pump 148, the deflation valve 150, and the volume measurement device 164. For example, the cuffs 112 and 114 are inflated and deflated via the associated conduits 142, the inflation valves 144 and 146, the air pump 148, and the deflation valve 150.

The first and second valves 144 and 146 are arranged between the first and second inflatable cuffs 112 and 114 and the air pump 148, respectively, and configured to regulate the flow of air from the air pump 148 by entirely or partially opening or closing their passageways. Further, the first and second valves 144 and 146 are configured to allow the flow of air from the first and second cuffs 112 and 114 to the defilation valve 150 to deflate the cuffs 112 and 114.

The air pump or compressor 148 is configured to supply air to the cuffs 112 and 114 to inflate them as necessary. The air pump 148 is configured in any type suitable for providing air to the cuffs.

The deflation valve 150 is connected to the first and second cuffs 112 and 114 through the conduits 142 and operates to selectively deflate the first and second inflatable cuffs 112 and 114.

The first and second sensors 152 and 154 are electrically connected to the controller 166 via the amplifiers 156 and 158 and the filters 160 and 162, respectively. The sensors 152 and 154 are incorporated into the cuffs 112 and 114, respectively. Upon inflation of the associated cuffs 112 and 114, the sensors 152 and 154 detect and produce electrical signals indicative of sensed pressures. In some embodiments, the detected signals are amplified by the amplifiers 156 and 158 and selectively filtered by the filters 160 and 162 before inputted to the controller 166.

The first and second sensors 152 and 154 can be constructed from any of a variety of devices that can sense changes in a physical condition and produce a related electrical signal. For example, piezoelectric elements, strain gauge, or optical assemblies are able to monitor and convert physical movements at the subject S to electrical signals. Any selected pressure measuring device is adaptable to a cuff mounting.

The amplifiers 156 and 158 operate to increase the power of the detected signals at the first and second sensors 152 and 154 before the signals are provided to the controller 166 for further processes.

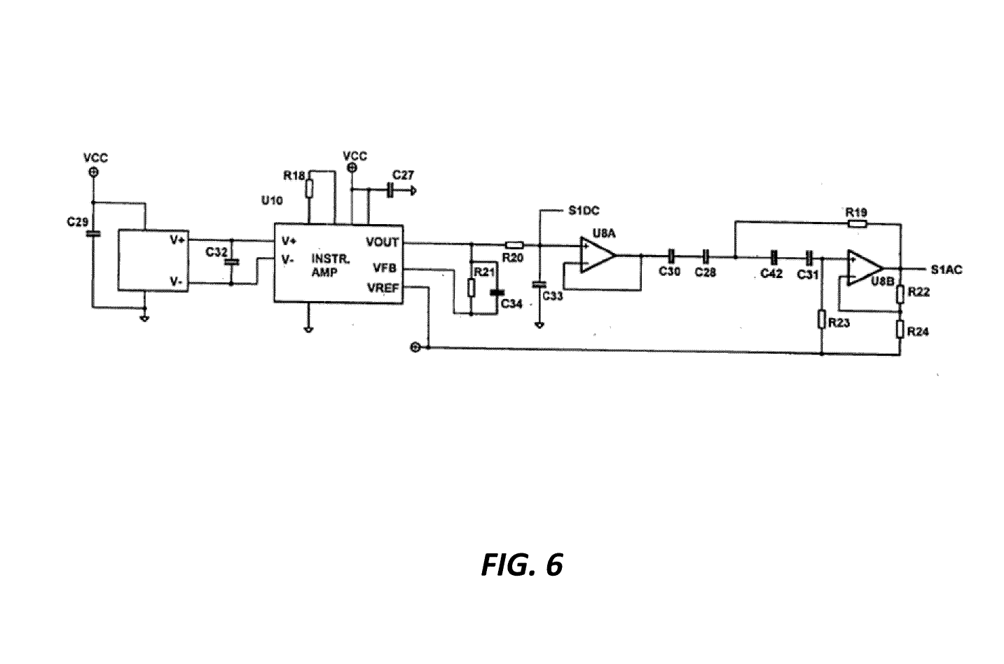

The filters 160 and 162 are selectively used to filter out AC components of the sensed blood flow signals. Along with DC components of the sensed blood flow signals, the AC components can be used to determine a systolic arterial pressure for a limb being monitored, as described herein. In some embodiments, the filters 160 and 162 are configured as band pass filters, which passes frequencies within a predetermined range at issue and attenuates frequencies outside that range. An example of the band pass filters are illustrated in more detail with reference to FIG. 6.

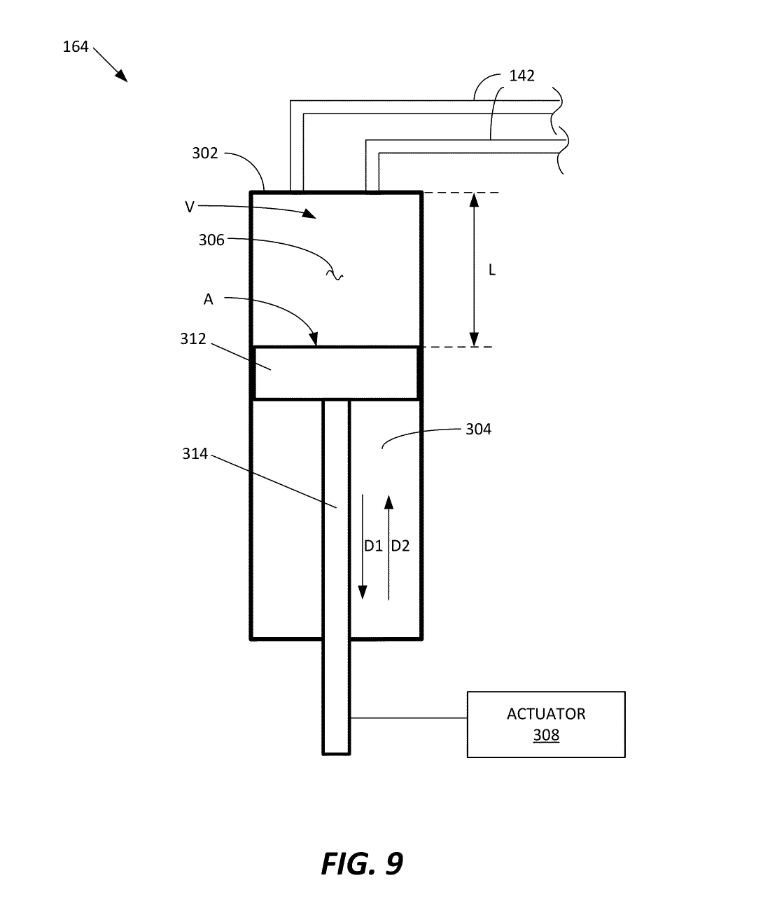

The volume measurement device 164 is configured as part of the volume measurement circuitry 136. As described, the volume measurement device 164 is configured to determine a pressure-to-volume relationship at the inflatable cuffs 112 and 114. The pressure-to-volume relationship is used in evaluating the vascular blood flow, such as a variety of venous blood flow tests. An example of the volume measurement device 164 is illustrated and described in more detail with reference to FIG. 9.

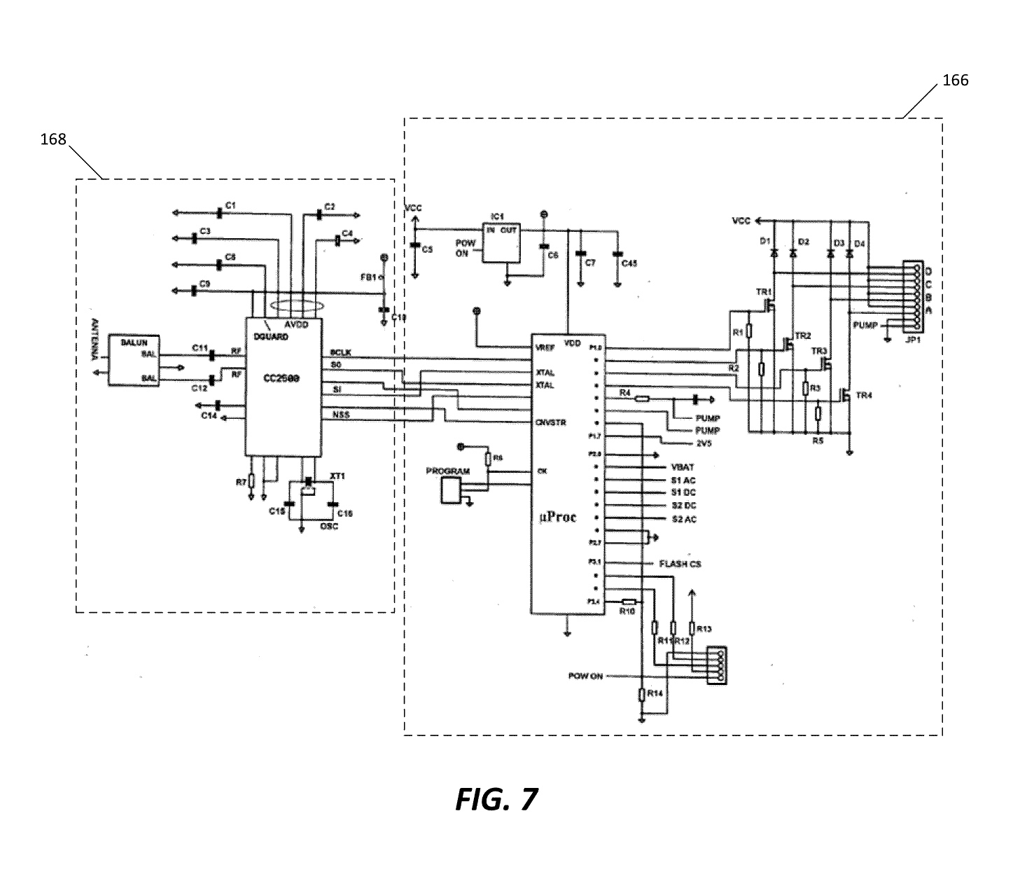

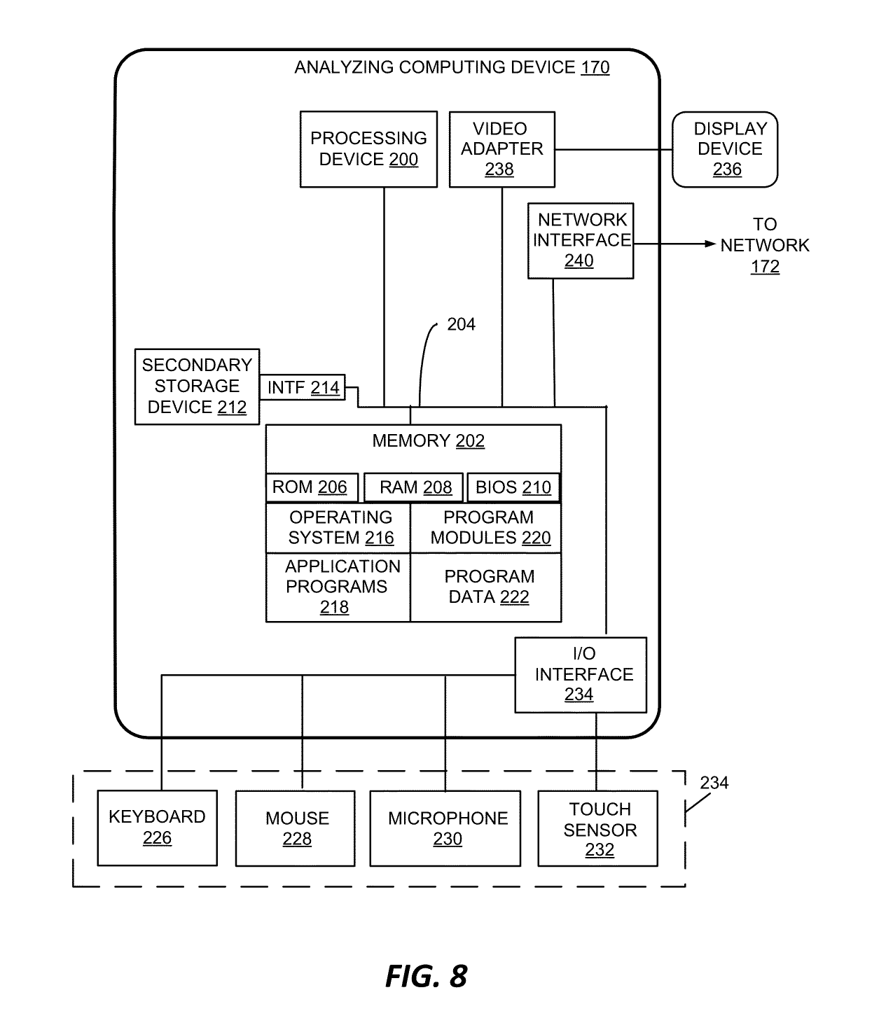

The controller 166 operates to control the components of the vascular measurement apparatus 102 and monitor the blood flow (e.g., blood pressure) of a limb at which the inflatable cuffs 112 and 114 are placed. For example, the controller 166 controls the inflation valves 144 and 146, the air pump 148, and the deflation valve 150 to manipulate the operation of the cuffs 112 and 114 as necessary for a variety of vascular blood tests (e.g., arterial blood flow tests and/or venous blood flow tests). The controller 166 can further control the operation of the volume measurement device 164 to obtain a pressure-to-volume relationship at the cuffs 112 and 114, as described herein. Further, the controller 166 receives the signals detected by the sensors 152 and 154 at the inflatable cuffs 112 and 114 for further processes. In some embodiments, the controller 166 can also be used to process the received blood flow signals to evaluate the blood flow at the limb monitored. In other embodiments, the controller 166 can send the signals to another processing unit, such as the analyzing computing device 170, for evaluation of the signals. In some embodiments, the controller 166 is configured as described in FIG. 8. An example of the controller 166 is illustrated and described in FIG. 7.

The communications device 168 provides an interface for the controller 166 to communicate with other computing devices via the network 172.

In some embodiments, the analyzing computing device 170 operates to communicate with the controller 166 and analyze the data obtained during diagnostic tests performed by the apparatus 102. For example, the analyzing computing device 170 is operative to perform the necessary interpolation of the test data and output the results obtained. In some embodiments, the analyzing computing device 170 can display the operation of the apparatus 102 in real-time and the results of the analysis. The analyzing computing device 170 can be incorporated within the apparatus 102 as part of the apparatus 102, in some embodiments. In some embodiments, the analyzing computing device 170 is configured as described in FIG. 8.

The communication network 172 communicates digital data between one or more computing devices, such as between the communications device 168 and the analyzing computing device 170. Examples of the network 172 include one or more of a local area network and a wide area network, such as the Internet. In some embodiments, the network 172 includes a wireless communication system, a wired communication system, or a combination of wireless and wired communication systems. A wired communication system can transmit data using electrical or optical signals in various possible embodiments. Wireless communication systems typically transmit signals via electromagnetic waves, such as in the form of optical signals or radio frequency (RF) signals. A wireless communication system typically includes an optical or RF transmitter for transmitting optical or RF signals, and an optical or RF receiver for receiving optical or RF signals. Examples of wireless communication systems include Wi-Fi communication devices (such as devices utilizing wireless routers or wireless access points), cellular communication devices (such as devices utilizing one or more cellular base stations), and other wireless communication devices.

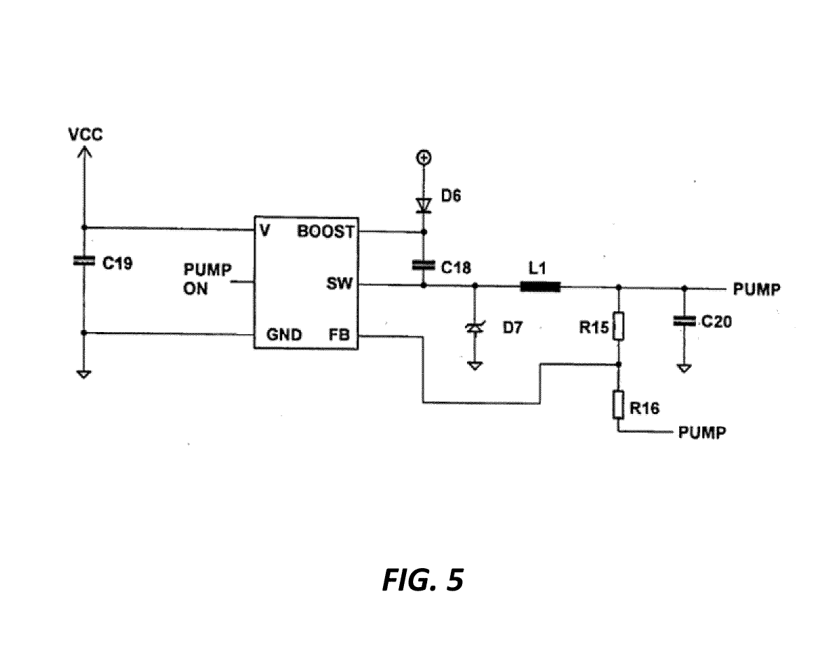

FIG. 5 is a circuit diagram of an example pump driver circuitry for operating the air pump 148 of FIG. 4. The pump driver circuitry is configured to inflate and deflate the first and second cuffs 112 and 114.

FIG. 6 is a circuit diagram of an example band pass filter used for the filters 160 and 162 of FIG. 4. The band pass filter as illustrated is configured to filter out AC components of the sensed blood flow signals within a predetermined range at issue and removes frequencies outside that range.

FIG. 7 is a circuit diagram of example controller 166 and communications device 168 of FIG. 4. As depicted, the controller 166 can include a microprocessor, which is associated with storage memory (e.g., RAM, ROM, flash) of suitable type and configuration. The controller 166 further includes drivers and input/output (I/O) circuitry to communicate with other components in the apparatus 102. In some embodiments, the controller 166 responds to a preprogrammed or programmable instruction set to control the operation of each of the inflatable cuffs 112 and 114 relative to the air pump 148 and monitor sensed pressures. In some embodiments, the communications device 168 can include a transceiver for wireless communication. Example transceivers include part number CC2500 available from Texas Instruments Inc., Dallas, Tex. In other embodiments, the communications device 168 establishes wired communication between the controller 166 and the analyzing computing device 170.

FIG. 8 illustrates an exemplary architecture of the analyzing computing device 170. In at least one embodiment, the architecture of the analyzing computing device 170 can also be similarly implemented in the controller 166. One or more computing devices, such as the type illustrated in FIG. 8, are used to execute the operating system, application programs, and software modules (including the software engines) described herein.

The computing device 170 includes, in at least some embodiments, at least one processing device 200, such as a central processing unit (CPU). A variety of processing devices are available from a variety of manufacturers, for example, Intel or Advanced Micro Devices. In this example, the computing device 170 also includes a system memory 202, and a system bus 204 that couples various system components including the system memory 202 to the processing device 200. The system bus 204 is one of any number of types of bus structures including a memory bus or memory controller, a peripheral bus, and a local bus using any of a variety of bus architectures.

Examples of computing devices suitable for the computing device 170 include a desktop computer, a laptop computer, a tablet computer, a mobile phone device such as a smart phone, or other devices configured to process digital instructions.

The system memory 202 includes read only memory 206 and random access memory 208. A basic input/output system 210 containing the basic routines that act to transfer information within computing device 170, such as during start up, is typically stored in the read only memory 206.

The computing device 170 also includes a secondary storage device 212 in some embodiments, such as a hard disk drive, for storing digital data. The secondary storage device 212 is connected to the system bus 204 by a secondary storage interface 214. The secondary storage devices and their associated computer readable media provide nonvolatile storage of computer readable instructions (including application programs and program modules), data structures, and other data for the computing device 170.

Although the exemplary environment described herein employs a hard disk drive as a secondary storage device, other types of computer readable storage media are used in other embodiments. Examples of these other types of computer readable storage media include magnetic cassettes, flash memory or other solid state memory technology, digital video disks, Bernoulli cartridges, compact disc read only memories, digital versatile disk read only memories, random access memories, or read only memories. Some embodiments include non-transitory media.

A number of program modules can be stored in secondary storage device 212 or memory 202, including an operating system 216, one or more application programs 218, other program modules 220, and program data 222. The data used by the computing device 170 may be stored at any location in the memory 202, such as the program data 222, or at the secondary storage device 212.

In some embodiments, computing device 170 includes input devices 224 to enable the caregiver to provide inputs to the computing device 170. Examples of input devices 224 include a keyboard 226, pointer input device 228, microphone 230, and touch sensor 232. A touch-sensitive display device is an example of a touch sensor. Other embodiments include other input devices 224. The input devices are often connected to the processing device 200 through an input/output interface 234 that is coupled to the system bus 204. These input devices 224 can be connected by any number of input/output interfaces, such as a parallel port, serial port, game port, or a universal serial bus. Wireless communication between input devices 224 and interface 234 is possible as well, and includes infrared, BLUETOOTH® wireless technology, 802.11a/b/g/n, cellular or other radio frequency communication systems in some possible embodiments.

In this example embodiment, a touch sensitive display device 236 is also connected to the system bus 204 via an interface, such as a video adapter 238. In some embodiments, the display device 236 is a touch sensitive display device. A touch sensitive display device includes sensor for receiving input from a user when the user touches the display or, in some embodiments, or gets close to touching the display. Such sensors can be capacitive sensors, pressure sensors, optical sensors, or other touch sensors. The sensors not only detect contact with the display, but also the location of the contact and movement of the contact over time. For example, a user can move a finger or stylus across the screen or near the screen to provide written inputs. The written inputs are evaluated and, in some embodiments, converted into text inputs.

In addition to the display device 236, the computing device 170 can include various other peripheral devices (not shown), such as speakers or a printer.

When used in a local area networking environment or a wide area networking environment (such as the Internet), the computing device 170 is typically connected to the network through a network interface, such as a wireless network interface 240. Other possible embodiments use other communication devices. For example, some embodiments of the computing device 170 include an Ethernet network interface, or a modem for communicating across the network.

The computing device 170 typically includes at least some form of computer-readable media. Computer readable media includes any available media that can be accessed by the computing device 170. By way of example, computer-readable media include computer readable storage media and computer readable communication media.

Computer readable storage media includes volatile and nonvolatile, removable and non-removable media implemented in any device configured to store information such as computer readable instructions, data structures, program modules, or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 170. Computer readable storage media is an example of a computer readable data storage device.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

In at least some embodiments of the computing devices, such as the controller 166 and the analyzing computing device 170, do not include all of the elements illustrated in FIG. 8.

FIG. 9 is a diagrammatic view of an example volume measurement device 164. In some embodiments, the volume measurement device 164 includes a barrel 302 and a plunger 304 defining a chamber 306 within the barrel 302. The volume measurement device 164 can further include an actuator 308.

The volume measurement device 164 operates to withdraw a predetermined volume of air contained in at least one of the first and second inflatable cuffs 112 and 114 and resupply the predetermined volume of air into the at least one of the first and second inflatable cuffs 112 and 114.

The barrel 302 provides a hollow container within which the plunger 304 is displaceable. The barrel 302 is configured to sealingly engage the plunger 304. In some embodiments, the barrel 302 is cylindrically shaped.

The plunger 304 is configured to move within the barrel 302 along a longitudinal direction D (i.e., either a first direction D1 or a second direction D2). In some embodiments, the plunger 304 includes a plunger head 312 and a plunger stem 314. The plunger head 312 is shaped and dimensioned to correspond to the inside of the barrel 302. The plunger head 312 slidably engages the inside of the barrel 302 while maintaining sealing between the plunger head 312 and the barrel 302.

The chamber 306 is defined by the plunger 304 (e.g., the plunger head 312) within the barrel 302. The chamber 306 is in fluid communication with the first and second inflatable cuffs 112 and 114 via the conduits 142 that pass through the ports 108 and 110. The chamber 306 contains air from at least one of the first and second cuffs 112 and 114 as the plunger 304 is operated by the actuator 308.

The actuator 308 operates to drive the plunger 304 in the direction D within the barrel 302. The actuator 308 can operate the plunger 304 in a first direction D1 to withdraw air from at least one of the first and second inflatable cuffs 112 and 114. Further, the actuator 308 can operate the plunger 304 in a second direction D2 opposite to the first direction D1 to refill the withdrawn air into the at least one of the first and second inflatable cuffs 112 and 114. The actuator 308 can be of any type, such as a hydraulic actuator, a pneumatic actuator, an electric actuator, and a mechanical actuator.

In some embodiments, the actuator 308 operates to move the plunger 304 within the barrel 302 in the first direction D1 to withdraw a predetermined volume V of air contained in at least one of the first and second inflatable cuffs 112 and 114 into the chamber 306, and move the plunger 304 within the barrel in the opposite direction (the second direction D2) to resupply the withdrawn air (the predetermined volume V of air) into the at least one of the first and second inflatable cuffs 112 and 114. In some embodiments, the volume measurement device 164 is configured such that the chamber 306 has a volume equal to the predetermined volume V when the plunger 304 is fully extended relative to the barrel 302 in the second direction D2. In other words, the maximum volume of the chamber 306 can be dimensioned to be the same as the predetermined volume V. In this case, when the plunger 304 is fully pulled by the actuator 308 to its maximum volume V, the predetermined volume V of air is withdrawn from at least one of the first cuff 112 and the second cuff 114. In certain examples, the predetermined volume V (i.e., the maximum volume of the chamber 306) is not more than 500 cc. In other embodiments, the predetermined volume V is not more than 100 cc. In yet other embodiments, the predetermined volume V is 20 cc. In yet other embodiments, the predetermined volume V is 10 cc.

In other embodiments, the volume measurement device 164 further includes a sensor module configured to determine a longitudinal displacement L of the plunger 304 relative to the barrel 302 so that the displacement L is used to calculate the volume V of air withdrawn from the first cuff 112 and/or the second cuff 114. The predetermined volume V of air can be calculated by detecting the longitudinal displacement L of the plunger 304 within the barrel 302 and multiplying the volume V by a projected area A of a surface of the plunger 304 (i.e., an end face of the plunger head 312) exposed to the chamber 306. In some embodiments, the sensor module is configured and arranged to detect a position of the plunger 304. For example, the sensor module 310 can include a position sensor configured as an absolute position sensor or a relative position sensor (i.e., a displacement sensor). The position sensor can be of any type suitable for measuring the absolute or relative position of the plunger 304. Examples of the position sensor include proximity sensor, rotary encoder, capacitive displacement sensor, ultrasonic sensor, Hall effect sensor, inductive non-contact position sensor, Laser Doppler Vibrometer, linear variable differential transformer, photodiode array, piezo-electric transducer, potentiometer, and string potentiometer. In other embodiments, the sensor module 310 detects a number of rotations of a driving end of the actuator 308 to determine the longitudinal displacement L of the plunger 304.

As the plunger 304 moves within the barrel 302 in the first direction D1 to withdraw the predetermined volume V of air contained in one of the first and second cuffs 112 and 114, a change in pressure is monitored at the one of the first and second cuffs 112 and 114. The pressure change can be detected by one of the first and second sensors 152 and 154 associated with the first and second cuffs 112 and 114, respectively.

The pressure-to-volume relationship is calculated from the predetermined volume V of air and the detected pressure difference from the associated cuff. In particular, the pressure-to-volume relationship represents a ratio of the predetermined air volume V to the detected pressure difference, or vice versa. The pressure-to-volume relationship reflects the pressure change at the monitored inflatable cuff (e.g., one of the cuffs 112 and 114) with respect to the volume change of the monitored cuff. The pressure-to-volume relationship correlates a pressure measured by an inflatable cuff into a volume, and thus allows converting between the volume and the pressure detected by the cuff.

For example, where the volume measurement device 164 has the chamber 306 with its maximum volume V of 20 cc and, the volume of air removed from the second cuff 114 is 20 cc when the plunger 314 is pulled to its maximum limit in the second direction D2 to withdraw air from the second cuff 114 (e.g., a sensing cuff). If the cuff pressure at the second cuff 114, which is monitored by the second sensor 154, changes from 30 mmHg to 15 mmHg as the air is removed from the second cuff 114, the pressure-to-volume relationship is calculated to be 20/15 (about 1.33) [cc/mmHg], which means that a volume changes about 1.33 cc for a change of pressure by 1 mmHg.

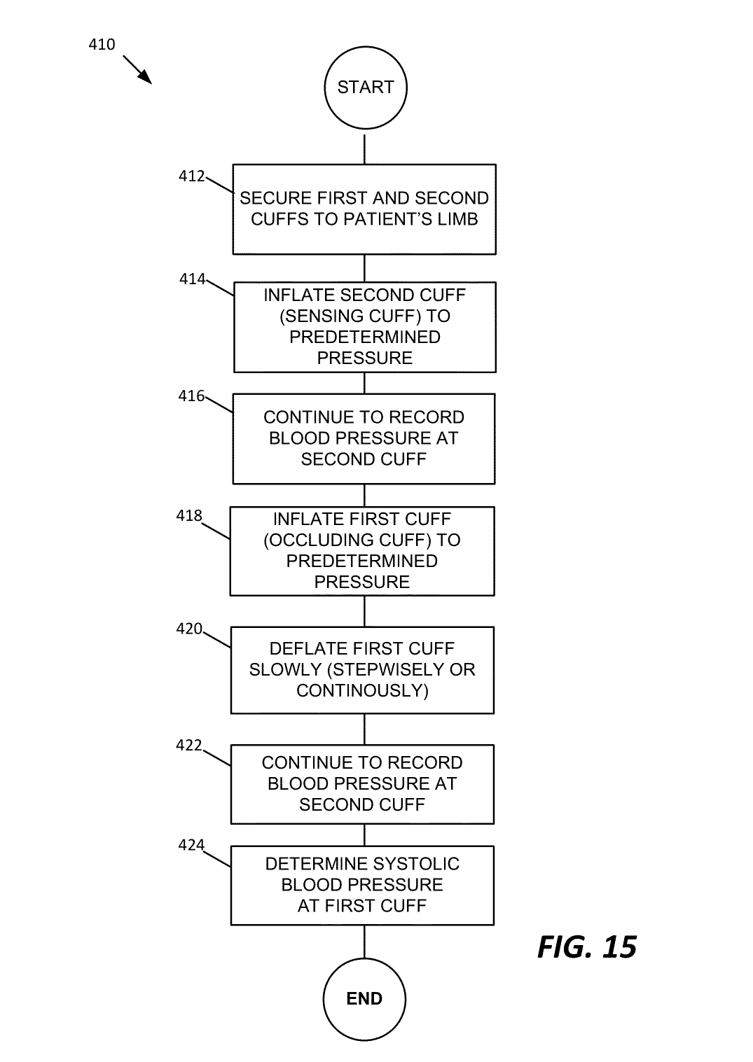

FIG. 10 is a diagram of an example system 100 for performing a variety of vascular measurements. In addition to the components as discussed in FIG. 1, the system 100 of this example further includes another set of ports, such as a third port 328 and a fourth port 330, which are configured to connect a third inflatable cuff 332 and a fourth inflatable cuff 334, respectively. Similarly to the first and second ports 108 and 110, the third and fourth ports 328 and 330 are not functionally distinguishable and configured to provide the same function and performance. Thus, the first, second, third and fourth ports 108, 110, 328 and 330 can be interchangeably used as necessary. Accordingly, the first, second, third and fourth ports 108, 110, 328 and 330 allow using the apparatus 102 for simultaneously performing both arterial blood flow test and venous blood flow test. For example, the first and second inflatable cuffs 112 and 114 connected to two different limbs of a test subject S to simultaneously perform arterial and venous blood flow tests by operating both the arterial blood flow measurement engine 104 and the venous blood flow measurement engine 106. As depicted in FIG. 10, the first and second ports 108 and 110 can be secured in the leg of the test subject S to perform a venous blood flow test, while the third and fourth inflatable cuffs 332 and 334 connected to the third and fourth ports 328 and 330 are secured in the arm of the subject S to perform an arterial blood flow test. Further, the four cuffs 112, 114, 332 and 334 associated with the four ports 108, 110, 328 and 330 can be secured to two different limbs of the subject S to simultaneously perform one of the arterial blood flow test and the venous blood flow test. For example, two of the four cuffs 112, 114, 332 and 334 can be secured to the subject's leg while the other two of the cuffs are secured to the subject's arm to obtain an ankle-brachial index (ABI), as described in FIG. 15.

Other than the number of ports and associated inflatable cuffs, the system 100 as illustrated in FIG. 10 shares the same concepts and features with the system 100 as described with reference to FIGS. 1-9. Thus, the description for the system 100 as described in FIGS. 1-9 is hereby incorporated by reference for the system 100 of FIG. 10.

Although the system 100 is illustrated in FIGS. 1-10 as including two or four ports and two or four associated inflatable cuffs, the system 100 can include any number of ports and associated inflatable cuffs, as necessary. Each of the ports and inflatable cuffs has the functionalities and features as described herein with respect to the ports 108, 110, 328 and 330 and the inflatable cuffs 112, 114, 332 and 334.

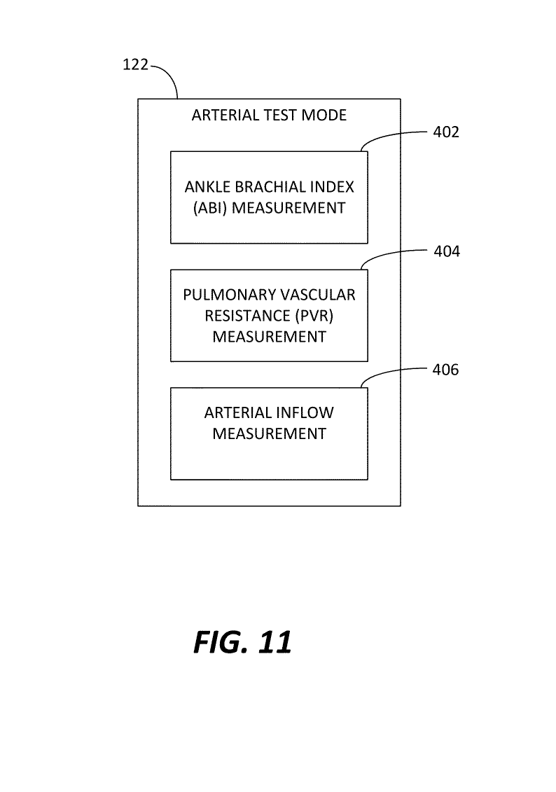

FIG. 11 illustrates examples of the arterial test mode 122 of FIG. 2. In some embodiments, at least one of an Ankle-Brachial Index (ABI) test 402, a pulmonary vascular resistance (PVR) measurement 404, and an arterial inflow (AI) measurement 406 is selectively performed in the arterial test mode 122. These example tests 402, 404 and 406 are not an exhaustive list of tests that can be performed in the arterial test mode 122. In other embodiments, the arterial test mode 122 includes other tests or measurements related to arterial blood flow.

The Ankle-Brachial Index (ABI), also known as Ankle Pressure Index (API) or Ankle Arm Index (AAI), is widely used to assess peripheral arterial disease. The ABI-test provides a well-documented, indirect method of comparing the relation of blood pressure in the arm to the blood pressure in the ankle and from which an assessment of arterial blood flow can be determined. Simply stated, ABI is the ratio of systolic blood pressure at the limbs (i e ankles/legs versus brachial/arms) and the general equation for determining ABI is as follows:

$$ABI = \frac{Ankle Systolic Blood Pressure}{Brachial Systolic Blood {Pressure}^{*}}$$

$${}^{*} Highest systolic pressure found in left or right arm$$

ABI has been shown to have a direct suggestive correlation to peripheral arterial disease (PAD) and also to have an inverse correlation to the risk of cardiovascular disease (CVD). PAD occurs when arterial vessels become occluded, partially occluded, or stenotic in the periphery. If left undiagnosed and/or untreated, the reduced flow condition(s) may lead to a higher risk of myocardial infarction, stroke, and cardiovascular mortality. While there are many causes of PAD, the most common cause is atherosclerosis. Atherosclerosis occurs with the build-up of deposits of fatty substances, for example, cholesterol, cellular waste products, calcium and other substances at the inner lining of an artery. This buildup is called plaque and usually affects large and medium-sized arteries. Some hardening of arteries occurs naturally as people grow older. Plaques can grow large enough to significantly reduce blood flow through an artery. The plaque can also become fragile and rupture. Plaques that rupture can cause blood clots to form that can further block blood flow and/or break off and travel to another part of the body. If either happens and blocks a blood vessel that feeds the heart, it causes a heart attack. If the clot blocks a blood vessel that feeds the brain, it causes a stroke. If the blood supply to the arms or legs is reduced, it can create difficulties in walking and in severe cases can eventually cause gangrene.

As shown in the table below, the risk of cardiovascular disease is inversely proportional to the ABI score. That is, the lower the ABI score, the greater risk of cardiovascular disease. Generally accepted ranges of ABI ratios and symptomatic conditions are shown in the table below. It is to be appreciated, however, that ABI values and ranges are not absolute and each individual's symptomatic condition can vary.

| ABI Ratio | Consideration |
|---|---|
| 0.96 or above | Generally Normal |
| 0.81-0.95 | Indicates mild, possibly asymptomatic disease |
| 0.51-0.81 | Indicates moderate disease |
| 0.31-0.50 | Usually indicates servere, multilevel occlusive |
| | disease |
| 0.30 or below | Severe disease. Usually indicates ischemic rest |
| | pain or tissue loss |

Source: The Cleveland Clinic, Department of Cardiovascular Medicine. Cleveland Ohio and Techniques in Noninvasive Vascular Diagnosis: Protocol and Procedures Guideline Manual. R. J. Daigle BA, RVT. Academy Medical Systems 1999. p. 134

ABI ratios are calculated by monitoring the arterial pressure of each of the right and left ankles and dividing the detected pressure by the highest brachial pressure found between either the left or right arm. Consequently for each exam, a Right ABI index value (i.e. right ankle pressure/highest arm pressure) and a Left ABI index value (left ankle pressure/highest arm pressure) is determined. The “highest” arm pressure is used in both calculations and the calculations are typically presented in mmHg (i.e. millimeters of Mercury). An example method of performing the ABI measurement 402 with the system 100 is illustrated and described with reference to FIGS. 15-21.

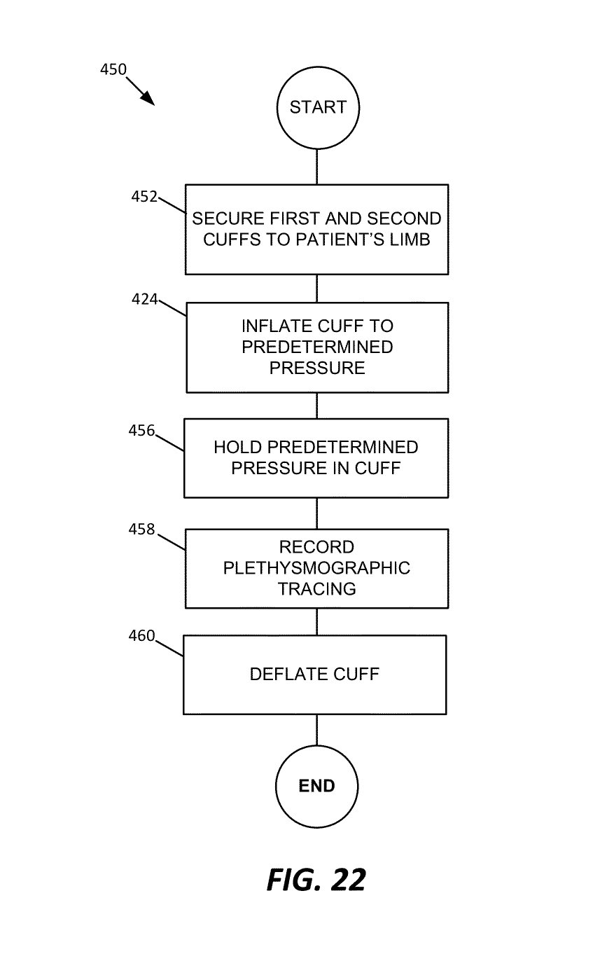

The pulmonary vascular resistance (PVR) measurement 404 detects vascular resistance, which represents the resistance to flow that must be overcome to push blood through the circulatory system. In particular, the PVR detects the resistance offered by the vasculature of the lungs. An example method of performing the PVR measurement 404 with the apparatus 102 is illustrated and described with reference to FIG. 22.

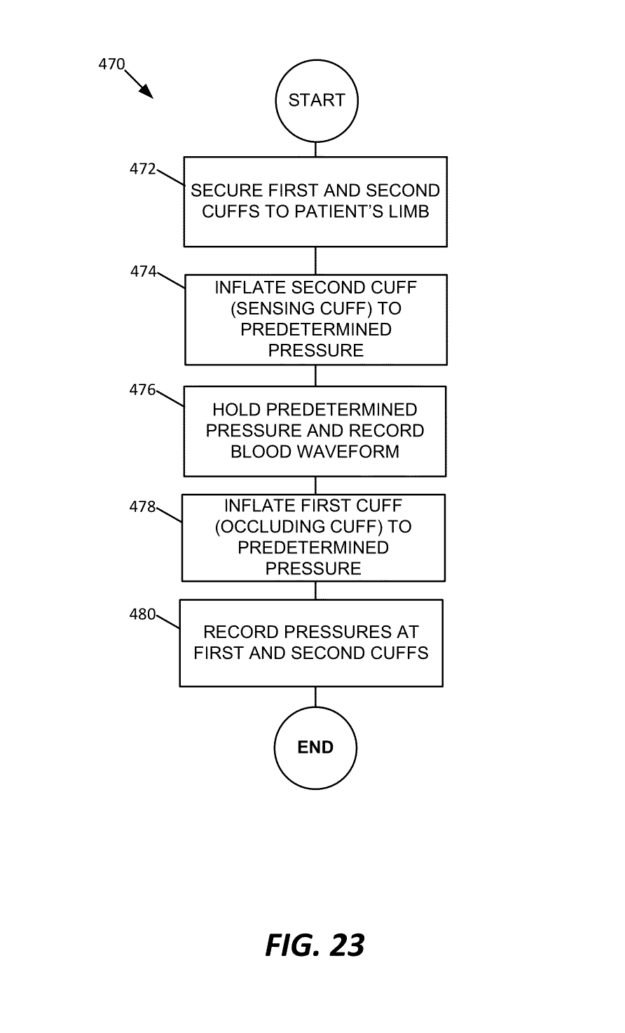

The arterial inflow (AI) measurement 406 is another test for determining peripheral vascular disease. An example method of performing the arterial inflow measurement 406 with the apparatus 102 is illustrated and described with reference to FIG. 23.

FIGS. 12-14 illustrate different placements of the inflatable cuffs 112 and 114 to perform the examples of the arterial test mode 122 of FIG. 11. In the examples of FIGS. 12-14, the first inflatable cuff 112 is used as an occluding cuff and the second inflatable cuff 114 is used as a sensing cuff. In general, the occluding cuff is located around the area of interest to obtain systolic arterial pressure. The sensing cuff is to be secured to any location distal to the occluding cuff.

FIG. 12 is a schematic view of an example placement of the inflatable cuffs 112 and 114 to perform the ABI test 402 or the PVR test 404. In some embodiments, to obtain a blood pressure of an ankle that is used for the ABI measurement, the first inflatable cuff 112 (i.e., the occluding cuff) is secured around the ankle of the subject S. The second inflatable cuff 114 (i.e., the sensing cuff) can be secured to any location of the subject S that is distal of the first inflatable cuff 112. In the depicted example, the second inflatable cuff 114 is located around the subject's foot.

In other embodiments, this arrangement of the inflatable cuffs can also be used to conduct the PVR test 404. In the PVR test 404, each of the inflatable cuffs 112 and 114 can operate independently and be used to record plethysmographic waveform one at a time.

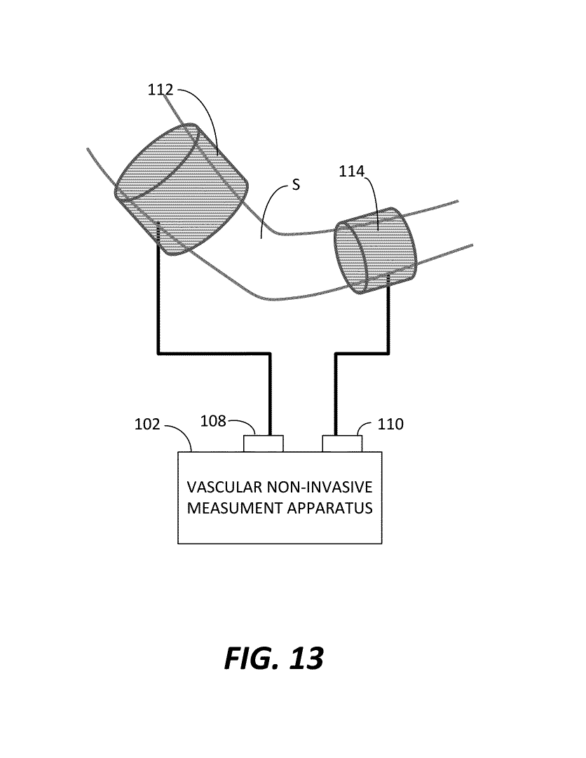

FIG. 13 is a schematic view of another example placement of the inflatable cuffs 112 and 114 to perform the ABI test 402. In some embodiments, to obtain a blood pressure of an arm (i.e., the brachial pressure) that is used for the ABI measurement, the first inflatable cuff 112 (i.e., the occluding cuff) is secured around the upper arm of the subject S. The second inflatable cuff 114 (i.e., the sensing cuff) can be secured to any location of the subject's arm that is distal of the first inflatable cuff 112. In the depicted example, the second inflatable cuff 114 is located around the lower arm of the subject.

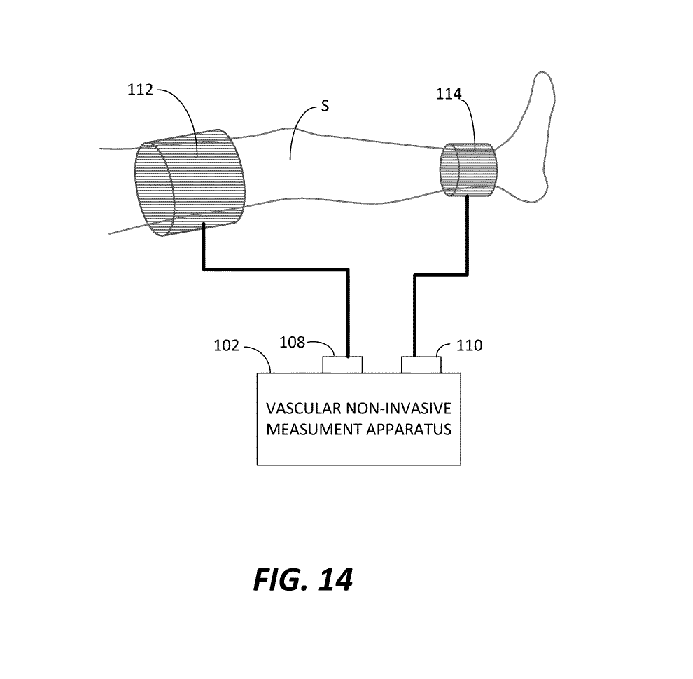

FIG. 14 is a schematic view of an example placement of the inflatable cuffs 112 and 114 to perform the arterial inflow measurement 406. In some embodiments, to obtain the arterial inflow of the subject S, the first inflatable cuff 112 (i.e., the occluding cuff) is arranged around the thigh of the subject S, while the second inflatable cuff 114 (i.e., the sensing cuff) is secured to a location of the subject's leg that is distal of the first inflatable cuff 112. In the depicted example, the second inflatable cuff 114 is located around the lower leg or ankle of the subject S.

FIG. 15 is a flowchart illustrating an example method 410 of performing the ABI measurement 402 with the system 100. In some embodiments, the method 410 includes operations 412, 414, 416, 418, 420, 422, and 424.

The system 100 provides an automatic system for performing the method 410 to conveniently and contemporaneously measure systolic arterial limb pressures. The detected pressures are used to determine a patient's ABI value. The system 100 was developed for ease of handling and operation by diagnostic personnel (e.g. nurses, medical technicians etc.), yet provides high sensitivity and accuracy. The measured ABI values and arterial pressures can be considered and reviewed by qualified diagnosticians for accuracy and utility relative to the subject's cardiovascular health condition. For certain patients, especially those with weak limb blood flow, meaningful data may be difficult to obtain.

Where the apparatus 102 includes four or more ports 108, 110, 328 and 330 that can connect four cuffs 112, 114, 332 and 334, the system 100 can provide a convenient system and assembly for obtaining a patient's ABI values by separately mounting the four cuffs 112, 114, 332 and 334 to a subject's arms and ankles The controller 166 operates to drive the air pump 148 and the deflation valve 150 to automatically inflate and deflate the cuffs, and determine the systolic blood pressure for each limb from signals obtained by the sensors associated with the cuffs.

The controller 166 and/or the analyzing computing device 170 evaluates the time/pressure data and during which the data is sampled and several indexed or addressable tables of sample values defining mean amplitude and derivative waveforms are derived. A variety of smoothing, fitting and scoring operations are performed on the sampled data to detect and remove artifacts (e.g. from the test procedure, electrical noise, subject motion) prior to determining relevant systolic pressure values for each monitored limb. The derived systolic limb pressure values are then used to determine right and left ABI values for a test subject.

In the depicted example, the method 410 is illustrated with the apparatus 102 having two ports (i.e., the first and second ports 108 and 110) and two associated inflatable cuffs (i.e., the first and second inflatable cuffs 112 and 114). It is to be appreciated that the method 410 is identically performed with the apparatus 102 having more than two ports and cuffs. For example, where the apparatus 102 includes four or more ports and cuffs, the apparatus 102 can monitor the subject's arm and ankle at the same time.

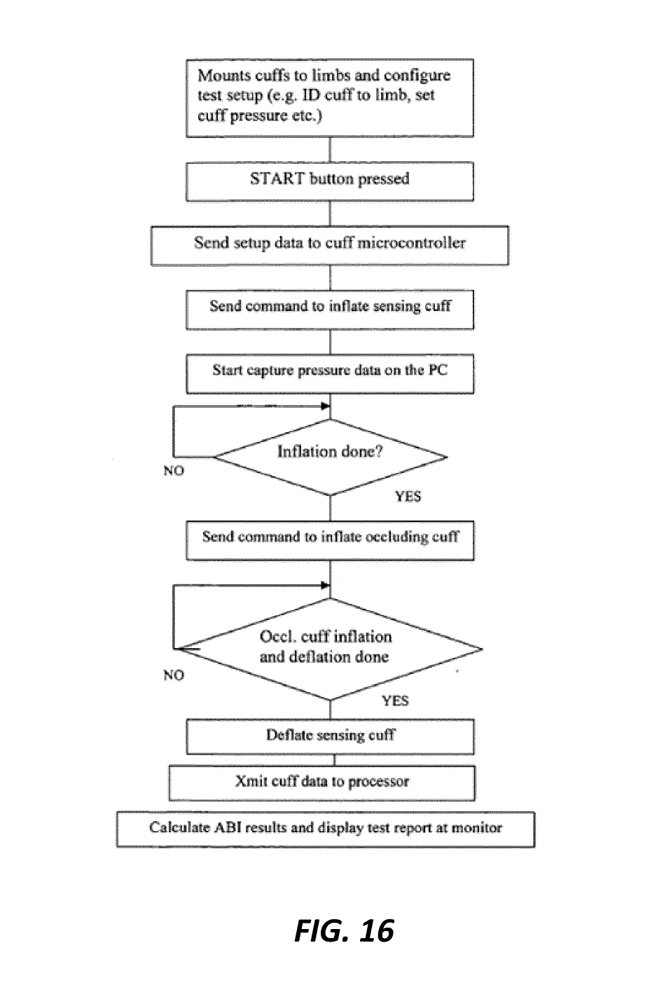

The method 410 typically begins at the operation 412. At the operation 412, the first and second inflatable cuffs 112 and 114 are secured to a limb of the test subject S. In some embodiments, the first and second inflatable cuffs 112 and 114 can be arranged as described in FIGS. 12 and 13. As described herein, each of the cuffs 112 and 114 are operative to expand and collapse with the movement of supplied and vented air. The controller 166 and/or the analyzing computing device 170 execute a micro-programmed operating and signal processing software instructions, as described with reference to FIGS. 16-19.

In the ABI measurement mode 402, the inflatable cuffs are operated as a sensing cuff and an occluding cuff. For example, one of the cuffs (e.g., the first cuff 112 (FIG. 1) or the third cuff 332 (FIG. 10)) is used as the sensing cuff, and the other (e.g., the second cuff 114 (FIG. 1) or the fourth cuff 334 (FIG. 10)) is used as the occluding cuff. It is to be appreciated that any of the inflatable cuffs (including the cuffs 112, 114, 332 and 334) connected to the apparatus 102 via the associated ports (including the ports 108, 110, 328 and 330) can be used as either a sensing cuff or an occluding cuff. In some embodiments, the cuffs with different sizes are used with the apparatus 102. For example, an inflatable cuff with a larger size can be used as an occluding cuff, and an inflatable cuff with a smaller size can be used as a sensing cuff.

In some embodiments, during the ABI measurement mode 402, each test is performed by first placing the patient S in a supine or horizontal position. The supine position places the limbs (e.g., arms and ankles) and the inflatable cuffs 112 and 114 at the same horizontal level as the heart. This position also tends to reduce motion artifacts and isolate systolic pressure variations to accurately reflect the subject's vascular condition. The inflatable cuffs 112 and 114 are next mounted to the subject's limbs. In some embodiments, the larger occluding cuff is mounted to a patient or subject's upper arm, and the smaller sensing cuff is mounted to the wrist or finger of the subject. In other embodiments, the larger occluding cuff is mounted to the subject's calf or leg in the region of the ankle, and the smaller sensing cuff is mounted to the ankle or toe. The controller 166 is configured to identify each cuff to the respective limb to which it is attached.

At the operation 414, the sensing cuff (e.g., the second cuff 114) is inflated to a predetermined pressure sufficient to assure intimate contact with the associated sensor (e.g., the second sensor 154). In some embodiments, the sensing cuff is inflated to about 30-40 mmHg. At this operation, the deflation valve 150 and the occluding cuff valve (i.e., the first valve 144) are closed, and the sensing cuff valve (i.e., the second valve 146) is opened, and the air pump 148 is engaged to inflate the sensing cuff.

At the operation 416, the controller 166 continues to monitor and record a blood pressure detected by the sensor (e.g., the second sensor 154) at the sensing cuff. In some embodiments, the operation 416 is performed concurrently with the other operations (e.g., the operations 414, 418, 420, 422, and 424) of the method 410.

At the operation 418, the occluding cuff (e.g., the first cuff 112) is then inflated to a predetermined pressure sufficient to occlude the artery and pulsed flow. In some embodiments, the occluding cuff is inflated to about 180 mmHg. At this operation, with the closing of the sensing cuff valve, the occluding cuff valve is opened and the air pump 148 operates to admit air into the occluding cuff.

At the operation 422, the occluding cuff is deflated slowly in an either continuous or stepwise fashion. At this operation, the controller 166 operates to open the deflation valve 150 and begins to deflate the occluding cuff. For example, the occluding cuff is deflated in an incremental step-wise fashion until normal pulsed flow returns to the limb. A generally linear deflation sequence with equal pressure drops at each step is presently performed. During deflation the AC and DC pressure signal components are sensed by the sensing and occluding cuffs and communicated to the controller 166.

At the operation 422, the controller 166 continues to monitor and record a blood pressure detected by the sensor (e.g., the first sensor 152) at the occluding cuff. In some embodiments, the operation 422 is performed concurrently with the other operations (e.g., the operations 414, 416, 418, 420, and 424) of the method 410. For example, blood pressures at the sensing and occluding cuffs are continuously monitored by the associated sensors throughout the operations of the method 410. The monitored blood pressures at the cuffs are used at the operation 424.

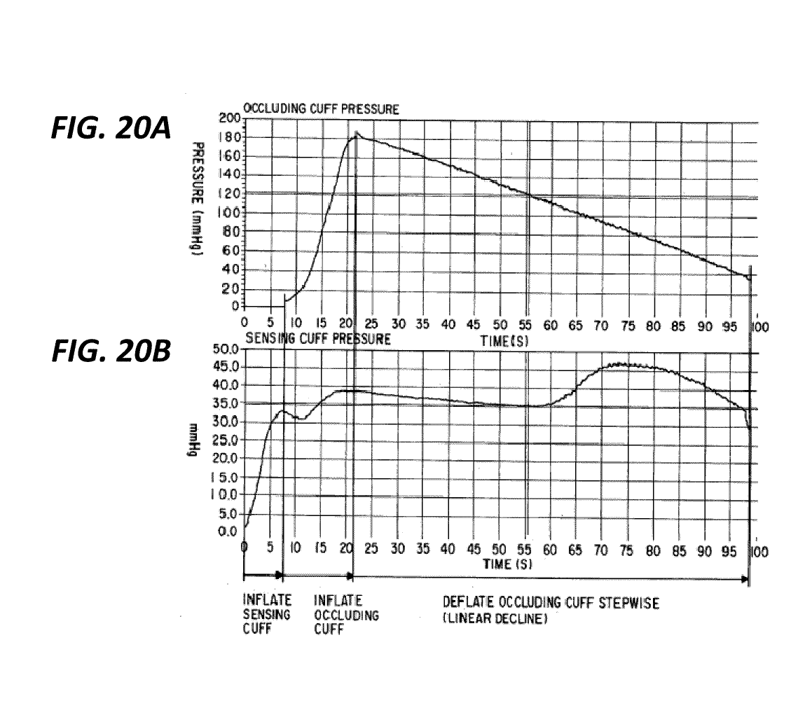

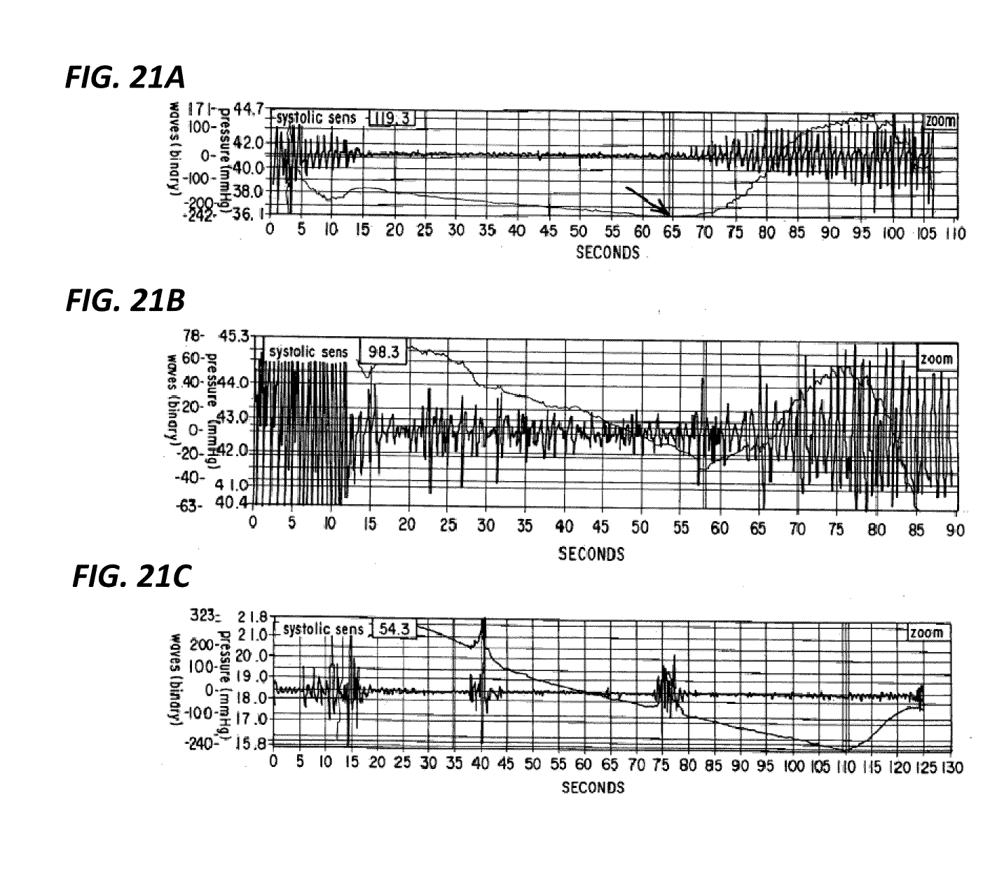

At the operation 424, the controller 166, either alone or with the analyzing computing device 170, operates to determine a systolic blood pressure at the first cuff (i.e., the occluding cuff) 112. When the first cuff 112 is secured around the subject ankle as illustrated in FIG. 12, the ankle pressure can be obtained by the operation of the controller 166. When the first cuff 112 is secured around the upper arm as illustrated in FIG. 13, the brachial pressure can be similarly obtained. These ankle and brachial systolic blood pressure measured are to be used to evaluate the ABI from the equation, as described above. An example operation of evaluating the ABI is illustrated and described with reference to FIGS. 20 and 21.

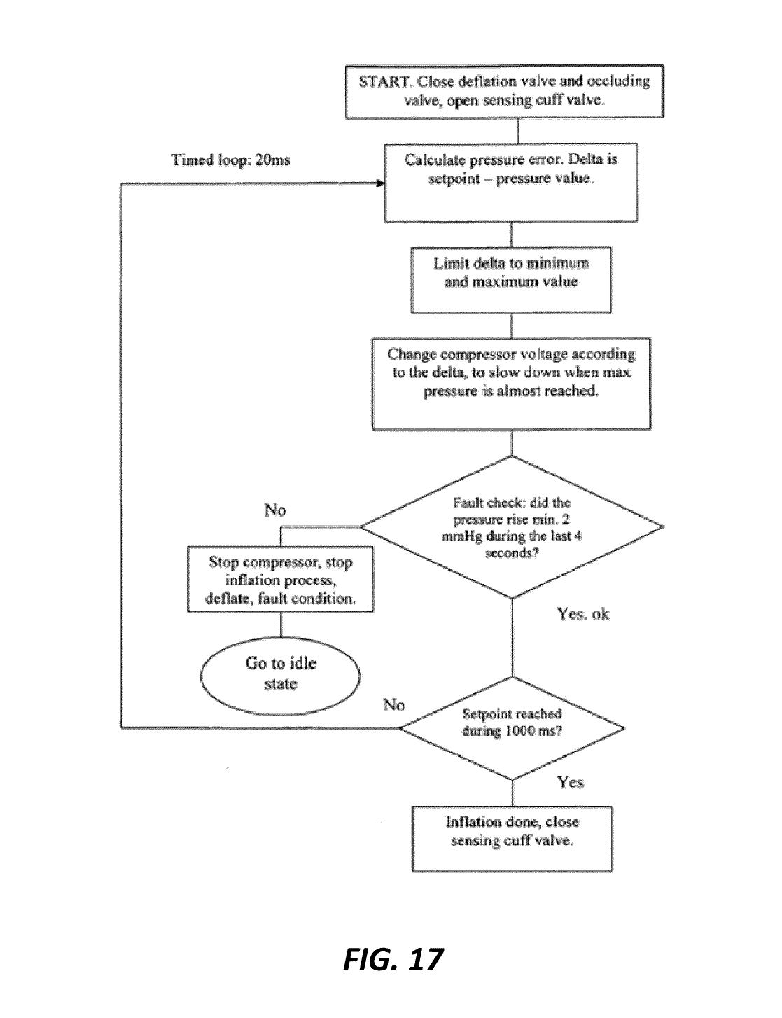

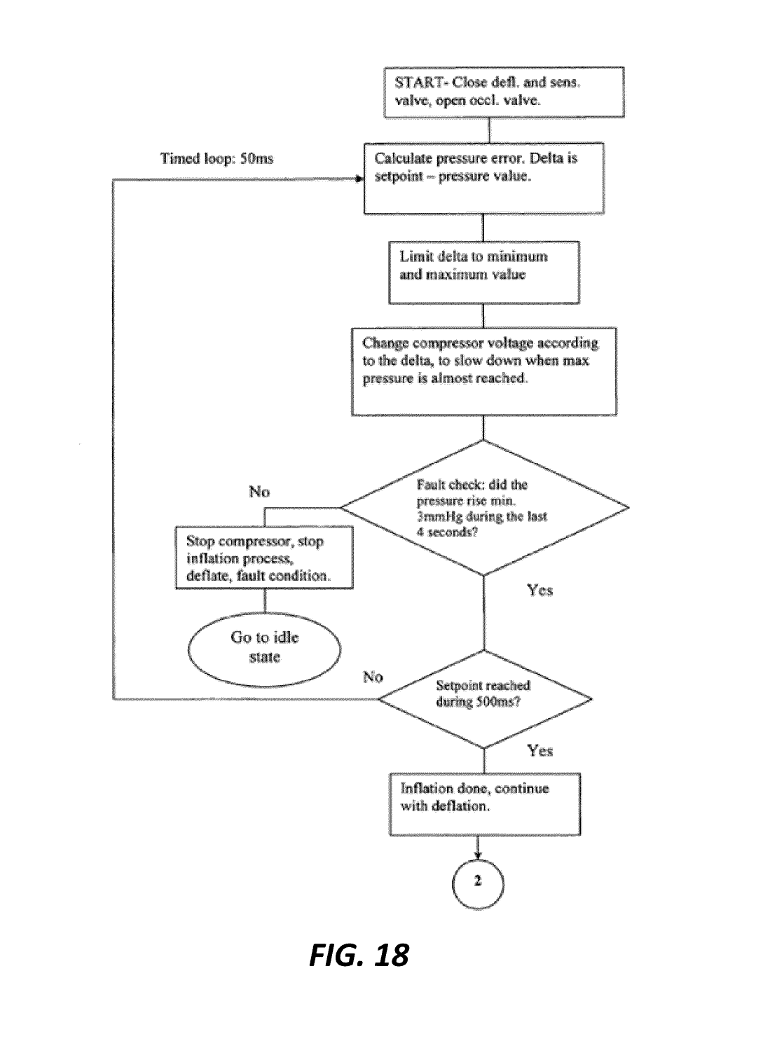

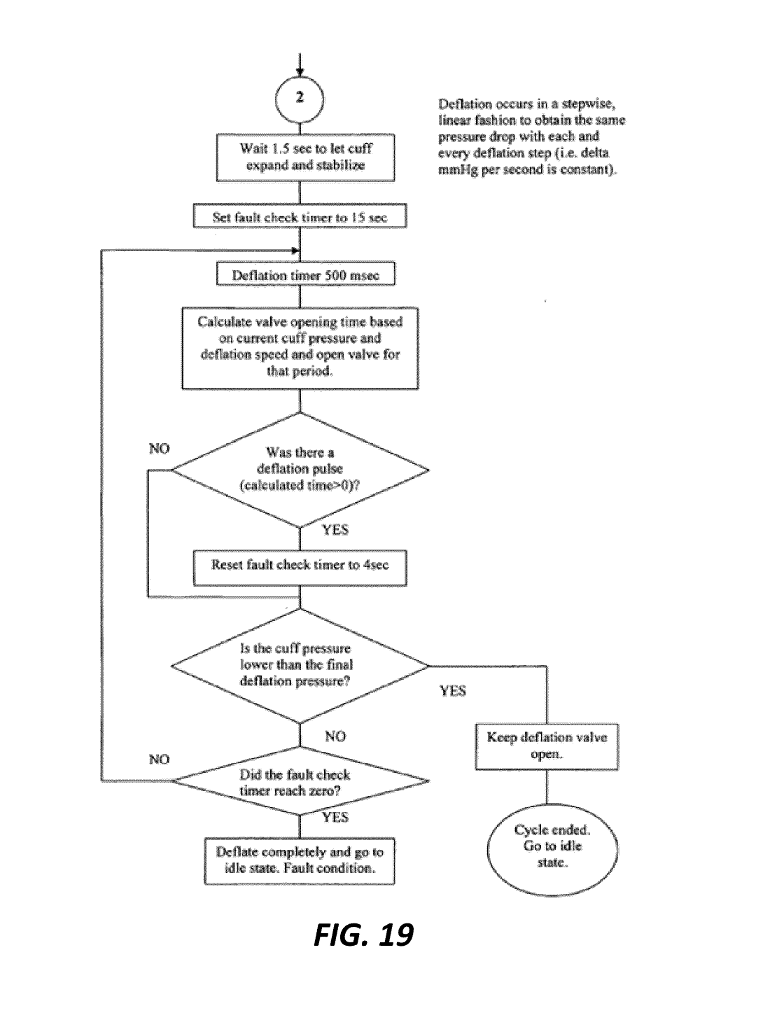

FIGS. 16-19 illustrate more detailed flowcharts illustrating the method 410 of FIG. 15, which is to perform the ABI measurement 402 with the system 100. In particular, FIG. 16 shows a flowchart to the general sequence of steps performed with each test of the ABI measurement 402 with the system 100. FIG. 17 is a flowchart illustrating the inflation of the sensing cuff in more detail. FIG. 18 shows a detailed flow chart to the inflation of the occluding cuff. FIG. 19 shows a detailed flowchart to the deflation sequence of the occluding cuff during the sensing phase of a test cycle and deflation of both cuffs upon completion of the test.

Referring to FIG. 16, the sensing cuff is first inflated to a pressure sufficient to assure intimate contact with the associated pressure sensor. The occluding cuff is then inflated to a pressure sufficient to occlude the artery and pulsed flow. The occluding cuff is then deflated in an incremental step-wise fashion until normal pulsed flow returns to the limb. A generally linear deflation sequence with equal pressure drops at each step is presently performed. During deflation the AC and DC pressure signal components are sensed by the cuffs and communicated to the controller.

Referring to FIG. 17, upon attaching the sensing and occluding cuffs to the test subject's limbs and placing the subject in a supine condition, a test “start” switch is initiated and the test setup data and instructions are sent to the controller. The deflation valve and the occluding cuff valve are closed, the sensing cuff valve is opened and the air compressor is engaged to inflate the sensing cuff to a set point pressure of approximately 30-40 mmHg. The controller is also enabled to record sensed pressure data or transmit the sensed pressure data to the analyzing computing device. The sensing cuff is inflated at a steady rate until just before the set point pressure. The air compressor is then slowed until the set point pressure is reached, when the air compressor is idled and the sensing cuff valve is closed.

Referring to FIG. 18, with the closing of the sensing cuff valve, the occluding cuff valve is opened and air is admitted into the occluding cuff. A maximum inflation or set point pressure is automatically established at the initiation of each test by the system software and is typically set at approximately 150% of the maximum pressure at which peak arterial pressure is sensed by the sensing cuff. A default, maximum inflation pressure (e.g. 250 mmHg) limited by the capacity of the air compressor or related equipment standards is also programmed into the apparatus 102. During each test, the occluding cuff is inflated to occlude the brachial artery in the arm and the femoral artery in the leg.

Assuming a nominal maximum pressure range of 180-250 mmHg, the pressure at the occluding cuff is monitored during inflation relative to the above range to regulate and slow the air compressor as the maximum set point pressure is approached. The sensed pulsed flow AC pressure signal at the sensing cuff is also monitored to determine the occlusion of flow in the limb. With a confirmation of occlusion at a pressure in the preset range, the controller stops the air compressor. After a few seconds to permit the pressures to stabilize, the controller opens the deflation valve and begins to deflate the occluding cuff in a stepwise manner, as illustrated in FIG. 19.

Referring to FIG. 19, during the deflation phase, the occluding cuff is particularly deflated in a stepwise fashion over a series of equal pressure drops. Pulse width modulated signals are continuously calculated and applied to control the open time of the deflation valve to achieve this end.

As the occluding cuff deflates, normal pulsed blood flow progressively returns to the limb. During each deflation step pulsed blow flow signals are progressively detected as the cuff pressure is released. The return of pulsed flow is better shown in the test data of FIG. 21A. With pulsed flow returning and the pressure at the occluding cuff falling below a preset final deflation pressure, the deflation valve is held open to release any remaining air from the occluding cuff. The air compressor is idled and the test is completed.

As air is released from the occluding cuff, the associated pressure sensor at the occluding cuff monitors the static cuff pressure. The pressure sensor at the sensing cuff contemporaneously senses the gradual return of pulsed blood flow to the limb as the arteries re-expand. The static DC pressure at the occluding cuff and the pulsed AC pressure at the sensing cuff are particularly monitored and contemporaneously coupled to a processor of the controller 166 and/or the analyzing computing device 170.

FIGS. 20-21 graphically represent data obtained and processed by the operations as illustrated in FIGS. 16-19. In particular, FIGS. 20A and 20B show exemplary pressure versus time waveforms for pressures sensed during a test of a limb at the occluding and sensing cuffs. FIGS. 21A-21C, in turn, depict composite plethsymographic waveforms for the limb under test at FIGS. 20A and 20B. In particular, FIG. 21A shows a plethsymographic waveform for limb data received from a test on a subject with no peripheral arterial disease (PAD), no artifacts and a mean arterial systolic pressure of 119.3 mmHg as determined by the software preprogrammed into the central processor. FIG. 21B shows a plethsymographic waveform for limb data received from a test on a subject with mild peripheral arterial disease (PAD), a very noisy center line wherein the artifacts may be due to calcification in the vessel, and a mean arterial systolic pressure of 98.3 mmHg. FIG. 21C shows a plethsymographic waveform for limb data received from a test on a subject with severe peripheral arterial disease (PAD), some motion artifacts at the center line, and a mean arterial systolic pressure of 54.3 mmHg.

In some embodiments, the composite waveform of FIGS. 21A-21C is generated by, for example, the analyzing computing device 170 upon processing the sensed DC and AC components of the pressure data signals using signal processing software preprogrammed into the analyzing computing device 170. Similar waveforms are obtained for each limb tested and from which “mean arterial pressures” are determined for each limb to compute a test subject's relevant ABI index values.

In some embodiments, the processor of the controller 166 and/or the analyzing computing device 170 process the data to determine the point in time where the static pressure at the sensing cuff reverts from a declining pressure slope to an inclining slope and nominal pulsed flow returns. The processor filters out extraneous pressure variations and slope changes to identify the primary or dominant slope change and related pressure at the waveform of FIG. 20A as the relevant systolic pressure. This pressure is used in the determination of the subject's ABI index values. For the test of FIGS. 20A and 20B, the processor determined the systolic pressure occurred approximately 55 seconds into the test. For the test of FIG. 21A, the systolic the pressure of 119.3 mmHg and slope reversion was determined to occur approximately 65 seconds into the test. The systolic pressure for both tests is determined from waveform data similar to that of FIG. 20A.

The test waveforms displayed at FIGS. 20A, 20B and 21A are representative of persons having generally healthy vascular systems and who did not move during their tests. The waveforms are therefore relatively free of artifacts. For a variety of reasons, such as less healthy subjects with occluded arteries or subjects that move or tense their muscles during a test, numerous pressure artifacts can be detected that produce several negative to positive and positive to negative slope changes in the sensed DC signals. FIGS. 21B and 21C depict test pressure waveforms for individuals with possible vascular blockage and/or who produced motion artifacts. In some embodiments, the analyzing computing device includes a signal processing software that is adapted to inspect each of these conditions and isolate the true negative to positive slope change and the related systolic pressure measured at that point by the occluding cuff.

FIG. 22 illustrates a flowchart of an example method 450 of performing the PVR measurement 404. In some embodiments, the method 450 includes operations 452, 454, 456, 458, and 460.

At the operation 452, the first and second inflatable cuffs 112 and 114 are secured to a limb of the test subject S. In some embodiments, the cuffs 112 and 114 are arranged as illustrated in FIG. 12. As described herein, each of the cuffs 112 and 114 are operative to expand and collapse with the movement of supplied and vented air. The controller 166 and/or the analyzing computing device 170 execute a micro-programmed operating and signal processing software instructions designed for performing the method 450.

In some embodiments, each of the first and second inflatable cuffs 112 and 114 can perform the PVR measurement individually and one at a time per limb. The first and second inflatable cuffs 112 and 114 are not to be used simultaneously on the same limb of the subject.

At the operation 454, one of the first and second cuffs 112 and 114 is inflated to a predetermined pressure. In some embodiments, the one of the first and second cuffs 112 and 114 is inflated to about 40 mmHg. The controller 166 operates to open the associated valve 144 or 146 and runs the air pump 148 to provide air into the one of the first and second cuffs 112 and 114 via the associated valve 144 or 146.

At the operation 456, the one of the first and second cuffs 112 and 114 is held at the predetermined pressure for recording at the operation 458. For example, the controller 166 operates to close the associated valve 144 or 146 to maintain the inflation of the one of the first and second cuffs 112 and 114.

At the operation 458, the controller 166 monitors and records plethysmographic tracing via the sensor 152 or 154 associated with the one of the first and second cuffs 112 and 114. In some embodiments, the monitored pressures are transmitted to the analyzing computing device 170 for evaluation.

At the operation 460, the one of the first and second cuffs 112 and 114 is deflated. For example, the controller 166 operates the deflation valve 150 to discharge air from the one of the first and second cuffs 112 and 114.

FIG. 23 is a flowchart of an example method 470 of performing the arterial inflow measurement 406. In some embodiments, the method 470 includes operations 472, 474, 476, 478, and 480.

At the operation 472, the first and second inflatable cuffs 112 and 114 are secured to a limb of the test subject S. In some embodiments, the cuffs 112 and 114 are arranged as illustrated in FIG. 14. As described herein, each of the cuffs 112 and 114 are operative to expand and collapse with the movement of supplied and vented air. The controller 166 and/or the analyzing computing device 170 execute a micro-programmed operating and signal processing software instructions designed for performing the method 450. In this test, the first cuff 112 is used as an occluding cuff, and the second cuff 114 is used as a sensing cuff

At the operation 474, the second cuff 114 (i.e., the sensing cuff) is inflated to a predetermined pressure. In some embodiments, the second cuff 114 is inflated to about 7 mmHg. The controller 166 operates to open the second valve 146 and runs the air pump 148 to provide air into the second cuff 114 via the valve 146.

At the operation 476, the second cuff 114 is held at the predetermined pressure and record a blood pressure at the second cuff 114 via the second sensor 154. For example, the controller 166 operates to close the second valve 146 to maintain the inflation of the second cuff 114, and obtain the blood pressure detected by the second sensor 154 at the second cuff 114.

At the operation 478, the first cuff 112 (i.e., the occluding cuff) is inflated to a predetermined pressure. In some embodiments, the first cuff 112 is inflated to about 60 mmHg. The controller 166 operates to open the first valve 144 and runs the air pump 148 to provide air into the first cuff 112 via the valve 144.

At the operation 480, the controller 166 operates to monitor and records blood pressures at the first and second cuffs 112 and 114. The blood pressures are detected by the first and second sensor 152 and 154 associated with the first and second cuffs 112 and 114, respectively. The monitored blood pressures can be transmitted to the analyzing computing device 170 for evaluation. In some embodiments, the operation 480 is performed concurrently with at least some of the other operations in the method 470.

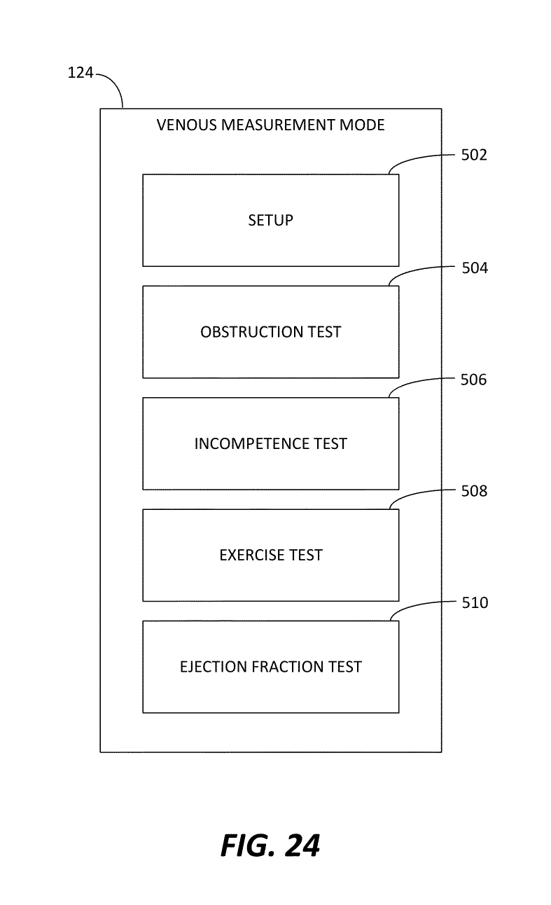

FIG. 24 illustrates example tests in the venous test mode 124 of FIG. 2. In some embodiments, at least one of a setup process 502, an obstruction test 504, an incompetence test 506, an exercise test 508, and an ejection fraction test 510 is selectively performed in the venous test mode 124. These example tests 502, 504, 506, 508 and 510 are not an exhaustive list of tests that can be performed in the venous test mode 124. In other embodiments, the venous test mode 124 includes other tests or measurements related to venous blood flow.

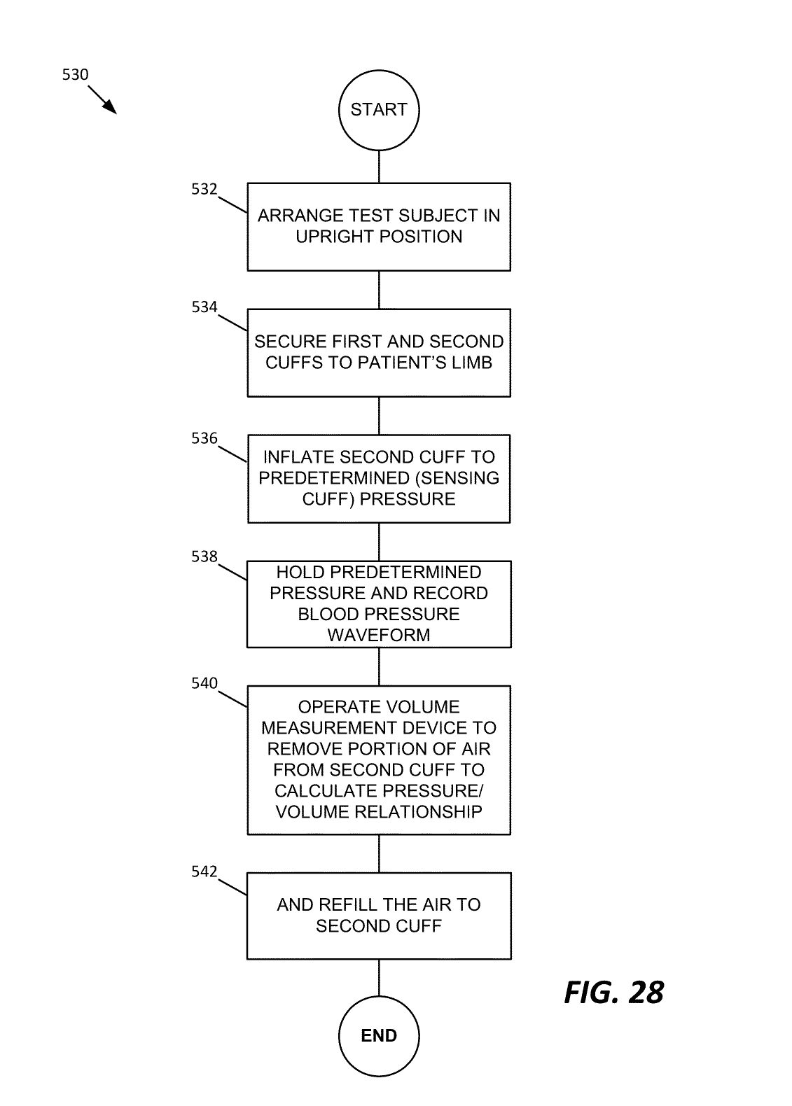

The setup process 502 is designed to arrange a test subject S, mount two of the cuffs 112, 114, 332 and 334 to the subject S, and measure the pressure-to-volume relationship. An example of the setup process 502 is illustrated and described with reference to FIG. 28.

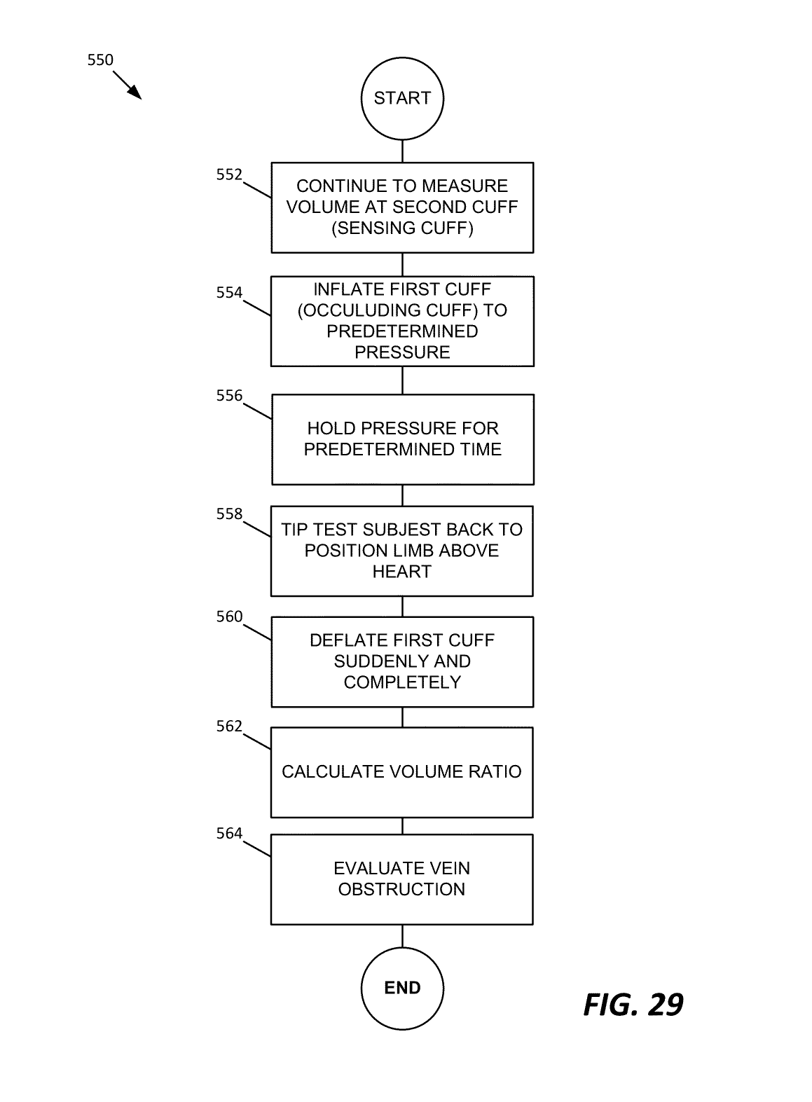

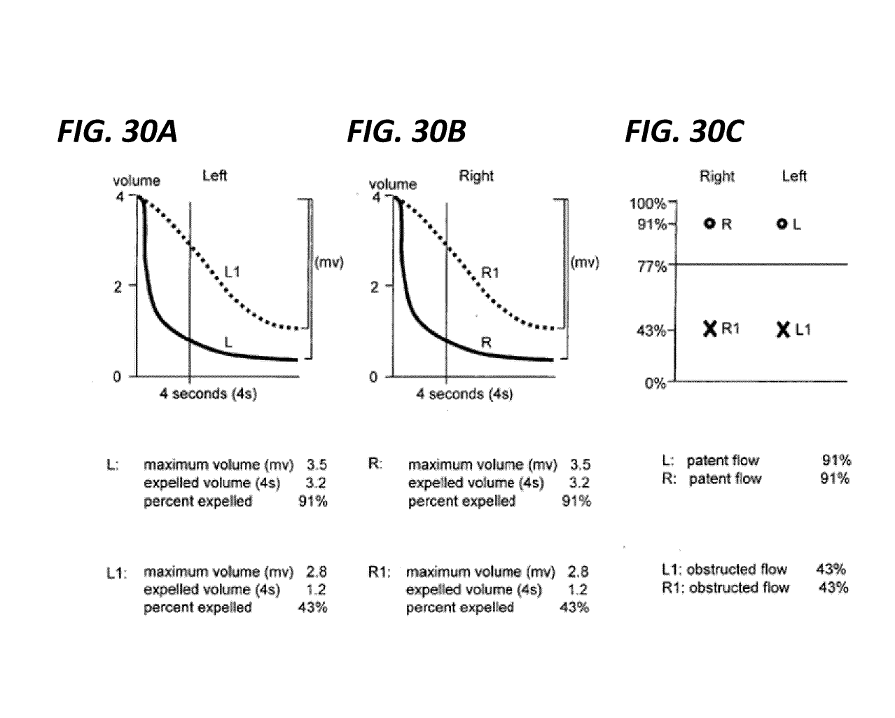

The obstruction test 504 is designed to evaluate any obstructions or blockages in the veins of the subject's limb. An example of the obstruction test 504 is illustrated and described with reference to FIGS. 29 and 30.

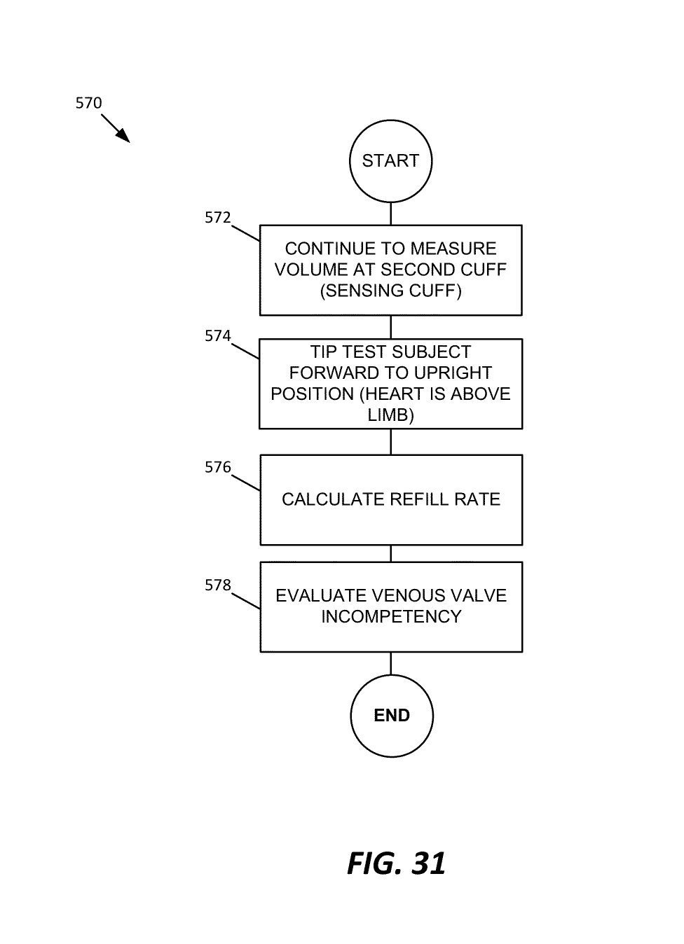

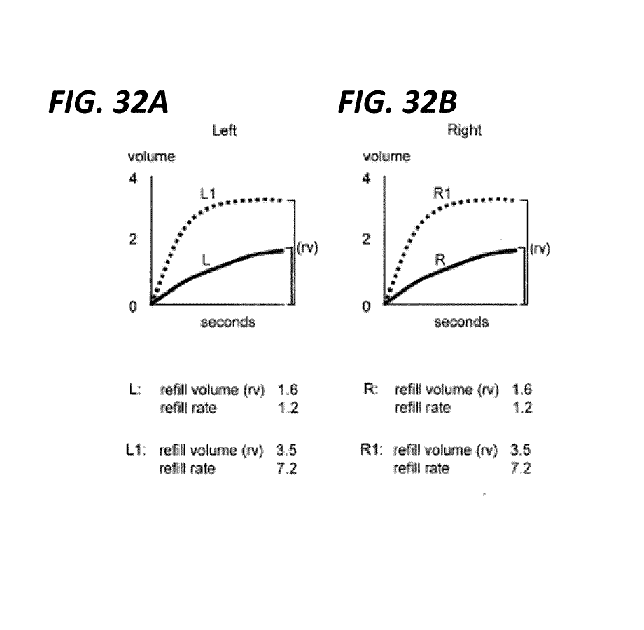

The incompetence test 506 is designed to measure incompetence of venous valves by testing how fast the limbs fill up with venous blood. An example of the incompetence test 506 is illustrated and described with reference to FIGS. 31 and 32.

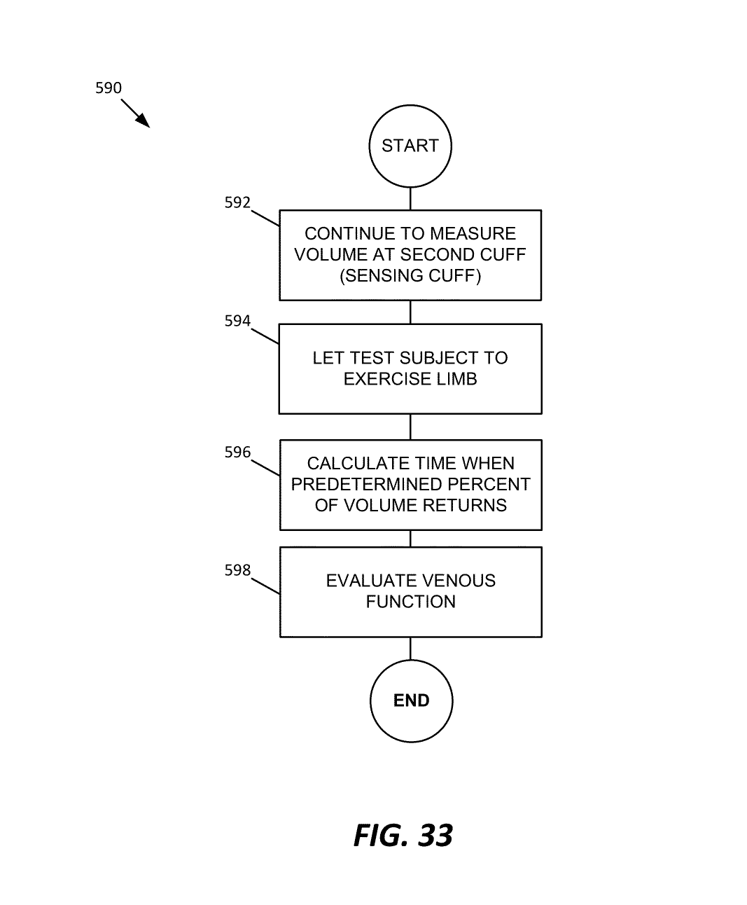

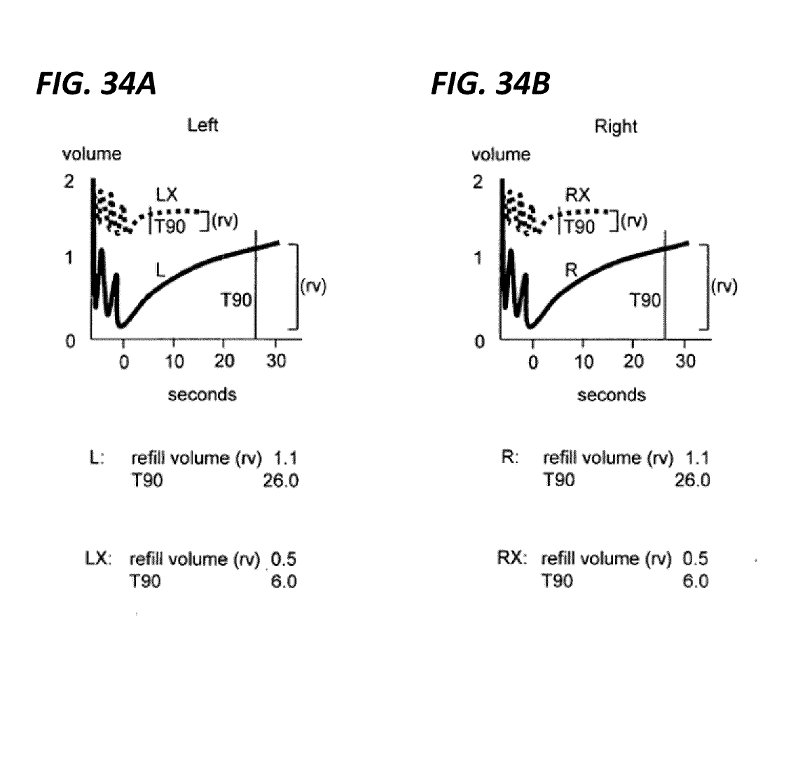

The exercise test 508 is designed to measure venous functions by detecting how much venous blood can be pumped as the subject moves the limb. An example of the exercise test 508 is illustrated and described with reference to FIGS. 33 and 34.

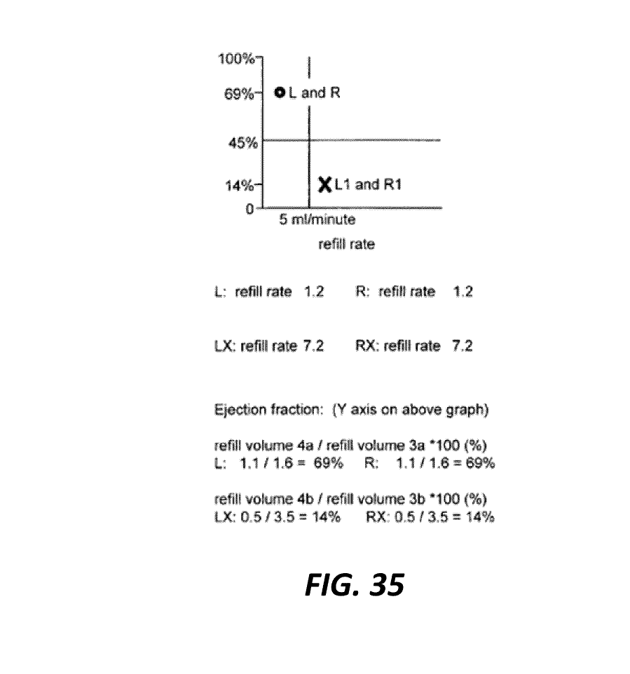

The ejection fraction test 510 is performed by combining data from the incompetence test 506 and the exercise test 508. An example of the ejection fraction test 510 is illustrated and described with reference to FIG. 35.

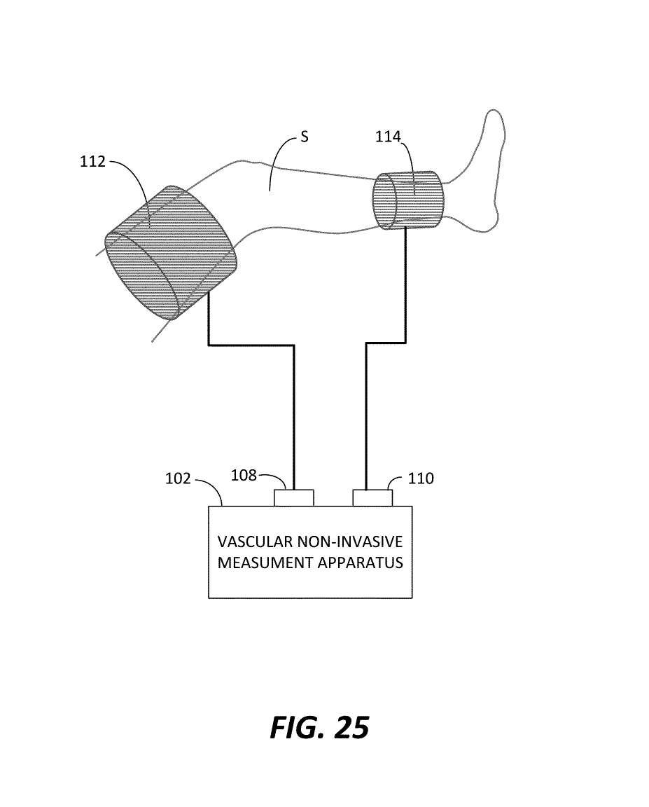

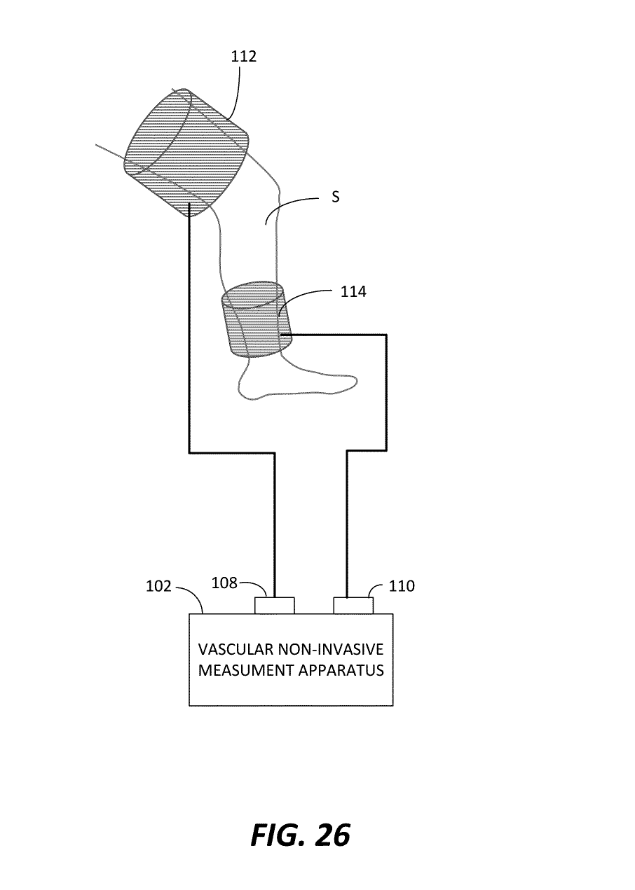

FIGS. 25 and 26 illustrate different placements of the inflatable cuffs 112 and 114 and leg positions to perform the examples of the venous test mode 124 of FIG. 11. In the examples of FIGS. 25 and 26, the first inflatable cuff 112 is used as an occluding cuff and the second inflatable cuff 114 is used as a sensing cuff. In general, the occluding cuff is located around the area of interest to obtain systolic arterial pressure. The sensing cuff is to be secured to any location distal to the occluding cuff

FIG. 25 is a schematic view of an example placement of the inflatable cuffs 112 and 114 to perform the obstruction test (i.e., the venous outflow test) 504. In some embodiments, the first inflatable cuff 112 (i.e., the occluding cuff) is secured around the thigh of the subject S, while the second inflatable cuff 114 (i.e., the sensing cuff) is secured around the ankle or lower leg of the subject S. As described below, the test subject S is tipped back to position the leg above the subject's heart.

FIG. 26 is a schematic view of an example placement of the inflatable cuffs 112 and 114 and the leg position to perform the incompetence test (i.e., the venous refill test) 506 and the exercise test 508. In some embodiments, the first and second inflatable cuffs 112 and 114 remain secured at the same location as shown in FIG. 25. However, the test subject S is tipped forward to an upright position so that the heart is positioned above the subject's leg.

As described below, the first inflatable cuff (i.e., the occluding cuff) 112 remains deflated in the incompetence test 506 and the exercise test 508. Therefore, in some embodiments, the incompetence test 506 and the exercise test 508 can be performed with the first inflatable cuff 112 removed.

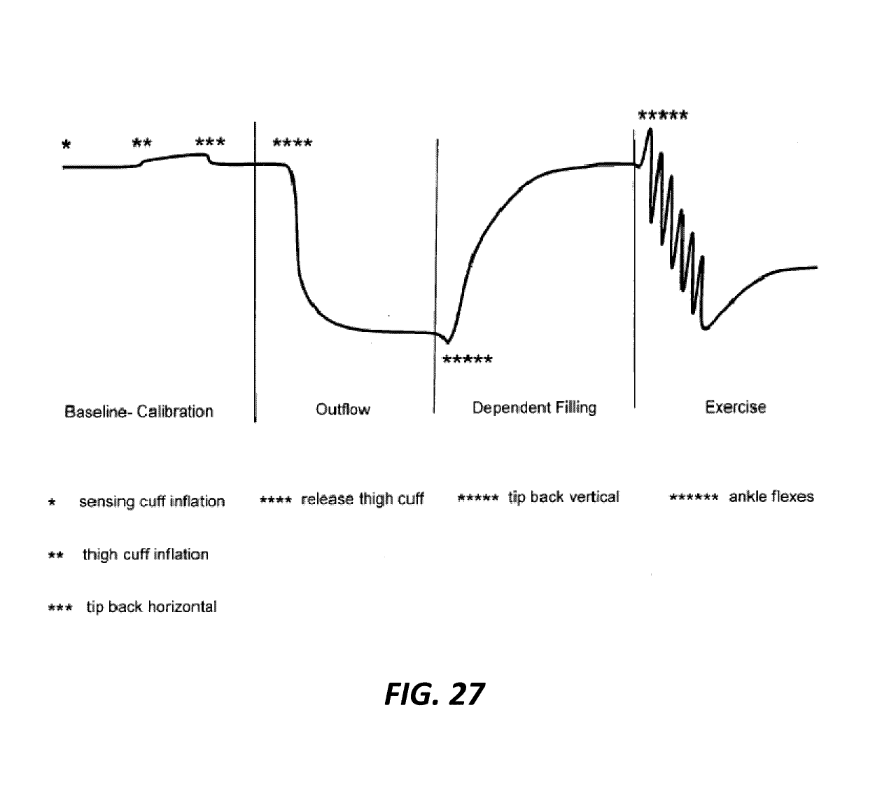

FIG. 27 shows a system operational timeline relative to the position of the subject and related venous blood flow volume measurements monitored by the system 100. The timeline schematically illustrates an automatic, plethysmographic method and sequence of steps that are typically performed in the venous test mode 124. The details of the timeline are described below with reference to FIGS. 28-35.

FIG. 28 is a flowchart of an example method 530 of performing the setup process 502 of FIG. 24. In some embodiments, the method 530 includes operations 532, 534, 536, 538, 540, and 542.

At the operation 532, the test subject S is arranged in an upright seated position. For example, the subject S can be seated on a chair.

At the operation 534, the sensing cuff (e.g., the second cuff 114 in FIG. 1) and the occluding cuff (e.g., the first cuff 112 in FIG. 1) are positioned around the subject's leg. In some embodiments, the sensing cuff is secured around the subject's calf or ankle, and the occluding cuff is secured around the subject's thigh, as illustrated in FIG. 25. Where two sensing cuffs and two occluding cuffs are used with the apparatus 102 as depicted in FIG. 10, the sensing cuffs are positioned around both legs of the subject S, and the occluding cuffs are positioned around both thighs. The sensing cuff is placed loosely to the leg (e.g., with a two finger space between the cuff and calf). Similarly, the occluding cuff is placed loosely fitted with a two finger space between the cuff and the thigh. The bottom of the sensing cuff can be located to nominally rest on the top of the foot. The tubing or conduits 142 are connected between the sensing cuff and the associated port (e.g., the second port 110 in FIG. 1) and between the occluding cuff and the associated port (e.g., the first port 108 in FIG. 1). In some embodiments, at this operation, the circumference of each leg is measured about 6 mm above the medial malleolus and record in mm, and the measurements can be entered in the computer at “venous” test program “circumference prompts”.

In some embodiments, the sensing cuff can be inflated to a predetermined pressure, such as 15 mmHg and then deflated to ensure that the sensing cuff is fitted to the subject's limb. Similarly, the occluding cuff can also be inflated to a predetermined pressure and then deflated to ensure the fitting of the occluding cuff to the subject's limb.

At the operation 536, the sensing cuff is inflated to a predetermined pressure. In some embodiments, the sensing cuff is inflated to about 4-8 mmHg. In other embodiments, the sensing cuff is inflated to about 5-6 mmHg.

At the operation 538, the controller 166 operates to hold the predetermined pressure at the sensing cuff. The controller 166 further records a blood pressure at the sensing cuff through the associated sensor (e.g., the second sensor 154). The recording operation can be performed concurrently with other operations in the method 530.

At the operation 540, the controller 166 operates the volume measurement device 164 to remove a predetermined volume V of air from the sensing cuff to establish the pressure-to-volume relationship. An example method of establishing the pressure-to-volume relationship is described above with reference to FIG. 9.

At the operation 542, once the pressure-to-volume relationship is determined, the volume measurement device 164 is operated to refill the predetermined volume V of air into the sensing cuff

FIG. 29 is a flowchart of an example method 550 of performing the obstruction test 504 of FIG. 24. In some embodiments, the method 550 includes operations 552, 554, 556, 558, 560, 562, and 564.

At the operation 552, the controller 166 continues to measure a blood pressure at the sensing cuff via the associate sensor after the air inflation process is completed in the method 530. In some embodiments, the controller 166 operates to display active pressure versus time in seconds tracings on a monitor display for the sensing cuff (or the sensing cuffs at the left and right legs). A baseline is reached when a predetermined pressure value programmed into a “setting” or test criteria parameter section of the controller program is reached.

At the operation 554, once a stable baseline condition is confirmed, the occluding cuff (e.g., the first cuff 112 in FIG. 1) is inflated to a predetermined pressure to occlude blood flow underneath the occluding cuff. In some embodiments, the occluding cuff is inflated to about 60 mmHg.

At the operation 556, the occluding cuff is held at the predetermined pressure for a predetermined period of time T1 (e.g., about 1-5 seconds). At this time, the sensing cuff tracings will rise due to blood being trapped in the extremity.

At the operation 558, the test subject is tipped back to position the limb above the heart. In some embodiments, after the predetermined period of time T1 after the occluding cuff inflation at the operation 556, the controller 166 and/or the analyzing computing device 170 produce an operator prompt “Tip patient back and press “OK”” to appear at the monitor of the apparatus 102. The subject is then tipped back until the subject's legs are positioned above the level of the heart. The subject is now positioned to begin “outflow plethysmography” with the object of looking for indicators of venous obstruction.

At the operation 560, the occluding cuff is deflated suddenly and completely. This causes the blood trapped in the lower leg to rush downward towards the heart. The sensing cuffs, in turn, continuously monitor and measure the amount of venous blood in the lower leg during and after the deflation period. At the operation 562, the controller 166 continues to measure the blood pressure underneath the sensing cuff, and the controller 166 and/or the analyzing computing device 170 calculate a volume ratio from the monitored blood pressure. For example, a volume measurement on the sensing cuff is taken at a predetermined time T2 after deflating the occluding cuff. In some embodiments, the predetermined time T2 is 4 seconds. The volume ratio is then calculated for the leg by the controller 166 and/or the analyzing computing device 170 by dividing the volume at the time T2 (e.g., 4 seconds) by the maximum volume from the volume flow tracings detected by the sensing cuff. A maximum flow volume is also obtained for the leg by measuring the volume in the sensing cuff before and after deflation. In calculating the volume ratio, the pressure-to-volume relationship is used to obtain the volume measurements from the blood pressure measurement by the sensor associated with the sensing cuff

At the operation 564, the volume ratio obtained at the operation 562 is used to evaluate vein obstruction or blockage. The evaluation can be performed automatically by the controller 166 and/or the analyzing computing device 170 and displayed on the display device of the apparatus 102 and/or the analyzing computing device 170. Examples of the data obtained and evaluation criteria are illustrated in FIG. 30.

FIGS. 30A-30C illustrate example test results of the obstruction test performed by the method 550 in FIG. 29. In particular, FIG. 30A shows an exemplary test result tracing indicative of monitored competence/function for patent flow (L, solid line) and obstructed flow (L1, dashed line) at a subject's left leg. FIG. 30B shows an exemplary test result tracing indicative of monitored competence/function for patent flow (R, solid line) and obstructed flow (R1, dashed line) at a subject's right leg. FIG. 30C shows a summary test result chart for the detected flows of FIGS. 30A and 30B relative to a pre-assigned 77% patent/obstructed ratio parameter, wherein the % ratios are computed from measured flow volumes 4 seconds after occluding cuff deflation±maximum volume measured at the sensing cuff before and after deflation and plotted relative to the “y” axis.

The data obtained after the occluding cuff deflates completely is displayed at the tracings shown at FIGS. 30A and 30B. A patent or normal flow condition is shown at the solid line tracings L and R and an obstructed flow condition is shown at the dashed line tracings L1 and R1.

Referring to FIG. 30C, the flow at a limb that is deemed “patent” if a ratio value greater than or equal to 77% is obtained. Flow at a limb is deemed “obstructed” if a ratio value less than 77% is obtained.

Conversely, once the occluding pressure on the L and R occluding cuffs is released, the venous blood trapped in the lower legs is able to move freely towards the heart limited only by the condition of the venous system and presence of any obstructions. If the blood can move freely, the volume in the sensing cuff decreases quickly (i.e. demonstrating “patent” flow). If the blood cannot move freely, the volume in the sensing cuff goes out slowly (i.e. demonstrating “obstructed” blood flow).

FIG. 31 is a flowchart of an example method 570 of performing the incompetence test 506 of FIG. 24. In some embodiments, the method 550 includes operations 572, 574, 576, and 578. As described above, the incompetence test 506 can be performed without the first inflatable cuff 112 (i.e., the occluding cuff) secured.

At the operation 572, the controller 166 continues to measure a blood pressure underneath the sensing cuff throughout the operations of the method 570. As described, the blood pressure is converted to volume measurement by the pressure-to-volume relationship.

At the operation 574, the test subject is tipped forward to an upright position so that the heart is positioned above the limb being tested. At the beginning of this test period, the subject's legs are initially held higher than the level of the heart and empty of venous blood. In some embodiments, the period is initiated with a display device of the apparatus 102 displaying a prompt “Press “OK”” causing the operator to bring the patient forward to an upright condition. The test subject is quickly brought forward while the sensing cuff measures the volume of blood that flows into the lower leg.

At the operation 576, the controller 166 calculates a refill rate. To calculate the refill rate, a volume value measured by the sensing cuff tracings is determined at a predetermined time T3 (e.g., 7.5 seconds), and a maximum value is obtained by measuring the volume in the sensing cuff before and after bringing the patient forward.

At the operation 578, the refill rate obtained at the operation 576 is used to evaluate venous valve incompetence. The evaluation can be performed automatically by the controller 166 and/or the analyzing computing device 170 and displayed on the display device of the apparatus 102 and/or the analyzing computing device 170. Examples of the data obtained and evaluation criteria are illustrated in FIG. 32.

FIGS. 32A and 32B illustrate example test results of the incompetence test performed by the method 570 in FIG. 31. In particular, FIG. 32A shows a test result tracing indicative of a monitored refill function for venous flow at a subject's left leg ((i.e. normal <5 ml/minute (solid line) and abnormal flow or >0.5 ml/minute (dashed line)). FIG. 32B shows a test result tracing indicative of monitored refill function for venous flow at a subject's right leg ((i.e. normal <5 ml/minute (solid line) and abnormal flow or >0.5 ml/minute (LX, dashed line)).

Referring to FIGS. 32A and 32B, L and R re-fill rate or flow volumes are measured at 7.5 seconds after establishing the subject in an upright condition. Blood flow is to be deemed normal if measured value is <5 ml/minute and abnormal if the flow is >5 ml/minute.

At the beginning of the incompetence test, the legs are higher than the level of the heart and empty of blood. When the patient is quickly brought forward, the venous blood attempts to rush back into the lower legs. Valves present in the venous system however prevent the blood from freely rushing back into the legs. In a limb that has normal valve control, the rate of refilling is very slow or usually less than 5 ml/minute. In a limb with damaged valves, the rate of refilling is very fast or usually more than 5 ml/minute.

FIG. 33 is a flowchart of an example method 590 of performing the exercise test 508 of FIG. 24. In some embodiments, the method 590 includes operations 592, 594, 596, and 598. As described above, the exercise 508 can be performed without the first inflatable cuff 112 (i.e., the occluding cuff) secured.

At the operation 592, the controller 166 continues to measure a blood pressure underneath the sensing cuff throughout the operations of the method 590. As described, the blood pressure is converted to volume measurement by the pressure-to-volume relationship.

At the operation 594, the test subject is instructed to exercise the limb being monitored. At the beginning of this test period, the subject is upright and the legs are full of venous blood. The sensing cuff is mounted to responsively continue to measure the amount of venous blood flow in the lower leg. In some embodiments, the period is initiated with the display device of the apparatus 102 displaying a prompt for the subject to perform 10 ankle flexes. Following the 10 ankle flexes, the subject remains still while the venous blood is allowed to refill with venous blood.

At the operation 596, a time is calculated when a predetermined percent of volume returns. To calculate the time, a volume value is obtained from the sensing cuff tracing when 90% of the maximum volume returns to the leg, and a maximum volume value is also obtained by measuring the volume at the sensing cuff before and after ankle flexes.

At the operation 598, the time obtained at the operation 596 is used to evaluate venous blood flow. The evaluation can be performed automatically by the controller 166 and/or the analyzing computing device 170 and displayed on the display device of the apparatus 102 and/or the analyzing computing device 170. Examples of the data obtained and evaluation criteria are illustrated in FIG. 34.

FIGS. 34A and 34B illustrate example test results of the exercise test performed by the method 579 in FIG. 33. In particular, FIG. 34A shows a test result tracing for the exercise period for normal (L, solid line) and abnormal (LX, dashed line) flow conditions indicative of monitored venous outflow function during the exercise period. FIG. 34B shows a test result chart for the exercise period for normal (R, solid line) and abnormal (RX, dashed line) flow conditions indicative of monitored venous outflow function during the exercise period.

Referring to FIGS. 34A and 34B, normal and abnormal blood flow conditions are determined in relation to the point in time or T90 when 90% of maximum volume returns to the legs. A normal condition is indicated if a time >25 seconds is determined and an abnormal condition is indicated if a time <25 seconds is determined.

By way of a generalized summary and during the exercise test period, the legs are full of venous blood at the beginning of the exercise test period. As the ankles are flexed blood is normally pumped out of the lower leg to the heart. After the ankle flexes, the venous blood pumped out attempts to rush back into the lower legs. Valves in the venous system however again prevent the blood from rushing back into the legs for a period that normally exceeds 25 seconds. An abnormal result is obtained if 90% of the blood is allowed to pass back into the legs in less than 25 seconds due to faulty valves.

FIG. 35 illustrates an example result of the ejection fraction test 510 of FIG. 24. In particular, FIG. 35 shows a summary “ratio” test result chart for ratios determined from the detected flows of FIGS. 30A, 30B, 34A and 34B relative to a pre-assigned 45% normal/abnormal ratio parameter, wherein the ratios are computed from the measured volume at the 90% level of the L and R refill volume tracings at the left and right legs depicted in FIGS. 29A and 29B (i.e. 1.1 normal and 0.5 abnormal) divided by the measured volume at the sensing cuff 4 seconds after occluding cuff deflation (i.e. 1.6 for L and R “patent” and 3.5 for LX and RX “obstructed flow”) conditions shown at FIGS. 30A and 30B.

Referring to FIG. 35, a “venous ejection fraction” is calculated by dividing the maximum volume measured at the venous exercise period (FIGS. 34A and 34B) by the maximum volumes measured during the dependent venous filling periods (FIGS. 32A and 32B). The resulting L and R and L1 and R1 ratios are depicted at FIG. 35. A normal condition is deemed to occur for a ratio >50% and an abnormal condition is deemed to occur for a ratio <50%.

By way of a generalized overview of the “ejection fraction”, if the dependent venous filling maximum volume value is deemed to exhibit a “full tank” and the venous exercise maximum volume is deemed to represent the amount of blood pumped from the lower legs during exercise. The ratio defines how much blood was pumped during leg exercise when each leg has its own maximum volume.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A system for determining blood pressure of a subject, the system comprising:
a first port configured to be connected to a first inflatable cuff, the first inflatable cuff coupled to a first pressure sensor;
a second port configured to be connected to a second inflatable cuff, the second inflatable cuff coupled to a second pressure sensor;
an inflation device configured to inflate at least one of the first and second inflatable cuffs;
a deflation device configured to deflate at least one of the first and second inflatable cuffs; and
a volume measuring device configured to withdraw a predetermined volume of air contained in at least one of the first and second inflatable cuffs and resupply the predetermined volume of air into the at least one of the first and second inflatable cuffs.

2. The system of claim 1, wherein the volume measuring device comprises:
a barrel;
a plunger defining a chamber within the barrel, the chamber being in fluid communication with at least one of the first and second inflatable cuffs through the first and second ports, and the plunger displaceable within the barrel; and
an actuator configured to move the plunger within the barrel.

3. The system of claim 2, wherein the actuator operates to move the plunger within the barrel in a first longitudinal direction to withdraw the predetermined volume of air contained in at least one of the first and second inflatable cuffs into the chamber, and move the plunger within the barrel in a second longitudinal direction opposite to the first longitudinal direction to resupply the withdrawn air into the at least one of the first and second inflatable cuffs.

4. The system of claim 3, wherein the predetermined volume of air is calculated by a longitudinal displacement of the plunger within the barrel, which is multiplied by a projected area of a surface of the plunger exposed to the chamber.

5. The system of claim 4, wherein the longitudinal displacement of the plunger is determined by a sensor module configured to detect a position of the plunger.

6. The system of claim 4, wherein the longitudinal displacement of the plunger is calculated based upon a number of rotations of a driving end of the actuator.

7. The system of claim 4, wherein the volume measuring device is configured to establish a pressure-to-volume relationship from the predetermined volume of air and a pressure difference of the at least one of the first and second inflatable cuffs, the pressure difference detected by at least of the first and second pressure sensors associated with the at least one of the first and second inflatable cuffs.

8. The system of claim 1, further comprising:
a monitoring device configured to monitor a pressure detected by at least one of the first and second pressure sensors.

9. An apparatus for evaluating vascular flow, the apparatus comprising:
a housing;
a plurality of ports arranged on the housing and configured to be connected to a plurality of inflatable cuffs, the plurality of inflatable cuffs coupled to a plurality of pressure sensors;
an inflation device configured to inflate at least one of the plurality of inflatable cuffs;
a deflation device configured to deflate at least one of the plurality of inflatable cuffs;
one or more processing devices within the housing; and
a computer readable storage device storing software instructions that, when executed by the one or more processing devices, cause the one or more processing devices to measure either arterial blood pressure of a test subject or venous blood pressure of the test subject from at least a part of the plurality of inflatable cuffs.

10. The apparatus of claim 9, wherein the software instructions further cause the one or more processing devices to simultaneously measure both of the arterial and venous blood pressures of the test subject from at least a part of the plurality of inflatable cuffs.

11. The apparatus of claim 9, further comprising:
a volume measuring device configured to withdraw a predetermined volume of air contained in at least one of the plurality of inflatable cuffs and resupply the predetermined volume of air into the at least one of the plurality of inflatable cuffs.

12. The apparatus of claim 11, wherein the volume measuring device comprises:
a barrel;
a plunger defining a chamber within the barrel, the chamber being in fluid communication with at least one of the plurality of inflatable cuffs through the plurality of ports, and the plunger displaceable within the barrel; and
an actuator configured to move the plunger within the barrel.

13. The apparatus of claim 12, wherein the software instructions further cause the one or more processing devices to calculate the predetermined volume of air by a longitudinal displacement of the plunger within the barrel, which is multiplied by a projected area of a surface of the plunger exposed to the chamber.

14. The apparatus of claim 13, wherein the longitudinal displacement of the plunger is determined by a sensor module configured to detect a position of the plunger.

15. The apparatus of claim 13, wherein the software instructions further cause the one or more processing devices to determine a pressure-to-volume relationship from the predetermined volume of air and a pressure difference of the at least one of the plurality of inflatable cuffs, the pressure difference detected by at least of the plurality of pressure sensors associated with the at least one of the plurality of inflatable cuffs.

16. A method of determining blood pressure of a subject, the method comprising:
arranging a test subject in a first position;
securing one of a plurality of inflatable cuffs to a limb of the test subject, the plurality of inflatable cuffs coupled to a plurality of pressure sensors;
inflating the one of the plurality of inflatable cuffs to a predetermined pressure;
recording a pressure from one of the plurality of pressure sensors coupled to the one of the plurality of inflatable cuffs;
withdrawing a predetermined volume of air contained in the one of the plurality of inflatable cuffs into a chamber of a volume measuring device by moving a plunger within a barrel of the volume measuring device in a first longitudinal direction;
recording a pressure change from the one of the plurality of pressure sensors coupled to the one of the plurality of inflatable cuffs; and
calculating a pressure-to-volume relationship from the predetermined volume and the pressure change.

17. The method of claim 16, further comprising:
resupplying the predetermined volume of air to the one of the plurality of inflatable cuffs from the chamber of the volume measuring device by moving the plunger within the barrel of the volume measuring device in a second longitudinal direction opposite to the first longitudinal direction.

18. The method of claim 16, further comprising:
securing a first cuff of the plurality of inflatable cuffs to a calf of the test subject;
securing a second cuff of the plurality of inflatable cuffs to a thigh of the test subject; and
evaluating a venous blood flow of the test subject between the first and second cuffs.

19. The method of claim 18, further comprising:
securing a third cuff of the plurality of inflatable cuffs to an upper arm of the test subject;
securing a fourth cuff of the plurality of inflatable cuffs to a wrist or finger of the test subject; and
evaluating an arterial blood flow of the test subject between the third and fourth cuffs.

20. The method of claim 19, further comprising:
securing a fifth cuff of the plurality of inflatable cuffs to a calf of the test subject;
securing a sixth cuff of the plurality of inflatable cuffs to an ankle or toe of the test subject; and
evaluating an arterial blood flow of the test subject between the fifth and sixth cuffs.
